(19) United States
(12) Patent Application Publication
(10) Pub. No.: US 2022/0111018 A1
(43) Pub. Date: Apr. 14, 2022

(54) COMPOSITIONS AND METHODS FOR INHIBITING YEAST INFECTIONS

(71) Applicant: University of Idaho, Moscow, ID (US)

(72) Inventors: Paul A. Rowley, Moscow, ID (US); Lance Fredericks, Garden City, ID (US); Angela M. Crabtree, Moscow, ID (US); Emily A. Kizer, Austin, TX (US)

(73) Assignee: University of Idaho, Moscow, ID (US)

(21) Appl. No.: 17/555,258

(22) Filed: Dec. 17, 2021

Related U.S. Application Data
(63) Continuation-in-part of application No. PCT/US2020/038696, filed on Jun. 19, 2020.
(60) Provisional application No. 62/684,743.

(51) Int. Cl. A61K 38/45; A61K 36/064; A61K 9/00

(52) U.S. Cl. CPC A61K 38/45; A61K 9/0036; A61K 36/064

(57) ABSTRACT

Killer toxins that may have activity against organisms, such as pathogenic organisms, and organisms that express the killer toxins are disclosed herein. The organism may be a recombinant organism or it may endogenously express a killer toxin. In some embodiments, a composition comprises one or more organisms, such as one or more Saccharomyces yeasts, and/or one or more killer toxins, such as K1, K2, and/or K1L toxins. In other embodiments, compositions comprise nucleic acids, vectors, host cells, or combinations thereof, comprising one or more killer toxins. Compositions can include one or more additional components, such as, but not limited to, a buffer, carrier, adjuvant, additional therapeutic, or combinations thereof. Kits that comprise one or more organisms and/or killer toxins are described. Uses of the organisms and/or killer toxins can include administration to a subject to treat an infection by one or more yeasts, such as Candida glabrata.

### CROSS REFERENCE TO RELATED APPLICATION

This application is a continuation-in-part of PCT Application No. PCT/US2020/038696, filed Jun. 19, 2020, which claims the benefit of U.S. Provisional Application No. 62/864,743, filed Jun. 21, 2019, all herein incorporated by reference.

### ACKNOWLEDGMENT OF GOVERNMENT SUPPORT

This invention was made with government support under Grant Nos. MCB 1818368 and BEACON DBI-093945 awarded by the National Science Foundation, and Grant No. INBRE 2P20GM103408 awarded by the National Institutes of Health. The government has certain rights in the invention.

### FIELD

The present disclosure relates generally to yeasts and proteins expressed by yeasts as inhibitors of pathogenic organisms, and to a method for treating or preventing yeast infections using the yeasts and/or proteins.

### BACKGROUND

Hospitalizations caused by opportunistic pathogens of the genus Candida result in healthcare costs of about $1.4 billion per year in the United States, and non-invasive candidiasis costs another $1.4 billion annually. After Candida albicans, C. glabrata is the second most prevalent cause of candidiasis in the U.S. Despite the availability of antifungal therapeutics, the Centers for Disease Control (CDC) considers C. glabrata an emerging threat to human health because of the increasing prevalence of drug resistance in the species.

Vulvovaginal candidiasis (VVC) affects approximately 70% of all women during their lifetimes and is currently treated using topical application of azole antifungal drugs. Most VVC cases are caused by C. albicans; however, 10-20% of cases are caused by non-albicans Candida yeasts that are frequently resistant to repeated courses of azole fungicides. Moreover, most non-albicans Candida isolates have been identified as C. glabrata. Topical flucytosine appears to be effective against these yeasts but is not recommended by the CDC and is not recommended for long-term use in general because of its toxicity to humans.

The emergence of drug resistant fungal pathogens and the cytotoxic side effects of existing antifungal therapeutics pose challenges to reducing human morbidity and mortality rates caused by fungal diseases. Increased azole resistance observed in C. glabrata and the ineffectiveness of azoles at lower pH levels necessitates the development of novel fungicides to treat VVC caused by C. glabrata.

### SUMMARY

Disclosed herein is a method for treating or preventing a yeast infection, such as in a subject. Methods for treating or preventing a yeast infection can comprise administering one or more killer toxins disclosed herein, to a subject, thereby treating or preventing the yeast infection in the subject. In some embodiments, the subject is an animal, such as a mammal, such as a human. Administering the killer toxin may comprise administering an organism that expresses the killer toxin. One or more killer toxins may be administered in combination with an organism that expresses one or more of the same or different killer toxins. A disclosed killer toxin-expressing organism may be, but is not limited to, a yeast, a recombinant yeast, a recombinant bacterium, a recombinant animal cell, a recombinant virus, or combinations thereof. In some embodiments, the yeast and/or the recombinant yeast is a member of genus Saccharomyces, such as S. cerevisiae or S. paradoxus.

The yeast infection may be vulvovaginal candidiasis, such as vulvovaginal candidiasis caused by a yeast of genus Candida. In some embodiments, the yeast of genus Candida is, or comprises, Candida glabrata.

In some embodiments, the administered killer toxin comprises a K1, K2, K28, K1us, K74, K21, K62, K45, K1L, KHR, or KHS killer toxin, or combinations thereof. In a specific, non-limiting example, the killer toxin is or comprises a K1 and/or K2 toxin. The K1 protein may comprise an amino acid sequence at least 80%, such as at least 85%, at least 90%, at least 95%, at least 96%, at least 97%, at least 98%, at least 99%, or substantially 100%, identical to any of SEQ ID NOs: 1-12. And/or the K2 protein may comprise an amino acid sequence at least 80%, such as at least 85%, at least 90%, at least 95%, at least 96%, at least 97%, at least 98%, at least 99%, or substantially 100%, identical to any of SEQ ID NOs: 13-18. In some embodiments, the K1L protein may comprise an amino acid sequence at least 80%, such as at least 85%, at least 90%, at least 95%, at least 96%, at least 97%, at least 98%, at least 99%, or substantially 100%, identical to SEQ ID NO: 38. In any embodiments, the method may comprise treating or preventing vulvovaginal candidiasis by administering a K1 protein, a K2 protein, a K1L protein, a yeast that expresses a K1 protein, a yeast that expresses a K2 protein, a yeast that expresses a K1L protein, or combinations thereof.

In certain embodiments, administering a killer toxin further comprises administering one or more pharmaceutically acceptable excipients, such as an adjuvant, a carrier, a buffer, another therapeutic, or combinations thereof. Additionally, or alternatively, the method may comprise administering a killer toxin, and/or an organism that expresses a killer toxin, in combination with one or more additional therapeutic agents, such as simultaneously or sequentially in any order.

Also disclosed herein are embodiments of a formulation for treating a yeast infection in a subject. In certain embodiments, the subject is an animal, such as a mammal, and may be a human. The formulation may be in the form of an ointment, cream, suppository, capsule, ovule, tablet, pill, solution, suspension, foam, film, gel, liposomal composition, or combinations thereof, and/or may comprises a killer toxin, an organism that expresses a killer toxin, or a killer toxin and an organism that expresses a killer toxin. The formulation may further comprise one or more pharmaceutically acceptable excipients, such as an adjuvant, a carrier, a buffer, another therapeutic, or combinations thereof. In certain embodiments, the formulation comprises a killer toxin, and/or an organism that expresses a killer toxin, in combination with another therapeutic.

In any embodiments of the formulation, a killer toxin-expressing organism may be, but is not limited to, a yeast, a recombinant yeast, a recombinant bacterium, a recombinant animal cell, a recombinant virus, or combinations thereof. In some embodiments, the yeast and/or the recombinant yeast is a member of genus Saccharomyces, such as S. cerevisiae or S. paradoxus.

In certain embodiments of the formulation, the yeast infection is vulvovaginal candidiasis, such as vulvovaginal candidiasis caused by a yeast of genus Candida. The yeast of genus Candida can be, but is not limited to, Candida glabrata.

In some embodiments of the disclosed formulation, the killer toxin comprises a K1, K2, K28, K1us, K74, K21, K62, K45, K1L, KHR, or KHS killer toxin, or combinations thereof. In a specific, non-limiting example, the killer toxin is a K1 and/or K2 toxin, wherein the K1 protein may comprise an amino acid sequence at least 80%, such as at least 85%, at least 90%, at least 95%, at least 96%, at least 97%, at least 98%, at least 99%, or substantially 100%, identical to any of SEQ ID NOs: 1-12. Additionally, or alternatively, the K2 protein may comprise an amino acid sequence at least 80%, such as at least 85%, at least 90%, at least 95%, at least 96%, at least 97%, at least 98%, at least 99%, or substantially 100%, identical to any of SEQ ID NOs: 13-18. In some embodiments, the K1L protein may comprise an amino acid sequence at least 80%, such as at least 85%, at least 90%, at least 95%, at least 96%, at least 97%, at least 98%, at least 99%, or substantially 100%, identical to SEQ ID NO: 38.

Also disclosed herein are embodiments of a composition comprising a killer toxin and/or an organism that expresses the killer toxin. In certain embodiments, the composition is for use in a method for treating a yeast infection in a subject. In a specific non-limiting example, the composition is administered to a subject having a yeast infection in an amount effective to treat the yeast infection in the subject. In certain embodiments, the composition is administered to a subject to treat a yeast infection in the subject caused by Candida glabrata. In a specific non-limiting example, the composition for use in treating a yeast infection in a subject comprises a K1, K2, and/or K1L killer toxin.

Also disclosed herein are embodiments of a vector comprising at least one killer toxin selected from the group comprising K1, K2, K28, K1us, K74, K21, K62, K45, K1L, KHR, KHS, or combinations thereof. Also disclosed are embodiments of a host cell comprising a disclosed vector.

Kits comprising a killer toxin, an organism that expresses a killer toxin, or combinations thereof also are disclosed. The kit may comprise a formulation as disclosed herein and an applicator suitable to administer and/or apply the formulation to a subject to treat or prevent a yeast infection. Suitable applicators include, but are not limited to, a tampon, a vaginal ring, a pillow, a puff, a sponge, an osmotic pump system, an applicator useful for administering a cream and/or ointment, an applicator useful for intravaginal administration of a capsule, ovule, tablet, pill, and/or suppository, or combinations thereof.

The foregoing and other objects, features, and advantages of the disclosure will become more apparent from the following detailed description, which proceeds with reference to the accompanying figures.

### BRIEF DESCRIPTION OF THE DRAWINGS

FIG. 1 provides digital images illustrating that the tested type strain of C. glabrata is broadly sensitive to a variety of killer toxins. Five pathogenic yeasts (C. glabrata, C. albicans, C. rugosa, C. auris, or C. tropicalis) were grown individually on agar plates. Nine different S. cerevisiae strains, each expressing a different killer toxin, were then inoculated into wells cut into the plates. S. cerevisiae strains (YJM1307, DSM-70459, OS169, OS78, OS294, OS299, MSC300C, OS40, CYC1172) and their respective toxins (K1, K1us, K62, K45, K74, Y8.5, K28, K21, K2, respectively) are shown in a grid (top left in Figure), with each grid square representing the respective well in the pictured agar plates. Killer toxin-expressing yeasts were assessed for the ability to prevent growth of the pathogenic yeast lawn or to stain the pathogenic yeast with methylene blue as an indicator of cell death. Growth inhibition was scored as the presence of a halo of growth inhibition. Arrowheads indicate smaller zones of growth inhibition or methylene blue staining. While 8 of the 9 toxins tested inhibited C. glabrata growth to some degree (only K62 did not inhibit C. glabrata), C. albicans growth was inhibited only by K1us, and K21 toxins; C. rugosa only by K1 and K21 toxins; C. auris only by the K1us toxin; and C. tropicalis only by the K1us toxin.

FIG. 2 provides a digital image illustrating that expression of the canonical K1 or a K1 variant was as effective at inhibiting C. glabrata growth as K1 expressed by S. cerevisiae strain BJH001, which comprises a totivirus-dsRNA satellites killer system. Ectopic expression of the K1 killer toxin effectively inhibits C. glabrata growth. A canonical K1 toxin or a K1 variant were separately cloned and expressed via multi-copy plasmids in the non-killer strain of S. cerevisiae strain BY4741. All strains tested produced zones of growth inhibition when assayed on methylene-blue agar plates at pH 4.6, regardless of toxin expression method.

FIG. 3 is a digital image that demonstrates that killer toxin activity can be characterized by the inhibition (such as the partial or complete inhibition) of yeast growth around the cells of yeasts that express the killer toxins and/or the staining of the surrounding inviable cells with the redox indicator methylene blue.

FIG. 4A is a digital image of an agar plate inoculated with C. glabrata strain ATCC_66032 and with wells cut therein, and depicts killer toxin challenge of strain ATCC_66032 with 73 diverse strains of killer toxin-expressing yeasts. The 73 strains were plated individually in the wells, and the level of inhibition of C. glabrata and/or induction of C. glabrata cell death by each of the 73 strains is shown.

FIG. 4B is a grid showing the location of each of the 73 strains in the agar plate of FIG. 4A. Each grid square provides the name of the strain located in the corresponding well of the agar plate.

FIG. 5A is a heatmap showing Saccharomyces yeast strains expressing killer toxins (including, but not limited to, K1, K1us, K62, K45, K74, K28, K21, K2) (columns; see FIGS. 5C-5D) that were used to challenge 135 diverse strains of C. glabrata (rows; see FIGS. 5E-5G) collected from both clinical and environmental niches from around the world. Each grid square shows the degree of growth inhibition of the given C. glabrata strain by the given killer toxin-expressing Saccharomyces strain, with darker squares indicating more robust growth inhibition.

FIG. 5B provides the key for interpreting FIG. 5A.

FIG. 5C provides the strain names of the killer toxin-expressing yeasts of a portion of the columns of FIG. 5A (the names of the strains of the leftmost and rightmost columns are shown in FIG. 5A).

FIG. 5D provides the strain names of the killer toxin-expressing yeasts of a portion of the columns of FIG. 5A (the names of the strains of the leftmost and rightmost columns are shown in FIG. 5A).

FIG. 5E provides the names of the C. glabrata strains of a portion of the rows of FIG. 5A (the names of the strains of the top and bottom columns are shown in FIG. 5A).

FIG. 5F provides the names of the C. glabrata strains of a portion of the rows of FIG. 5A (the names of the strains of the top and bottom columns are shown in FIG. 5A).

FIG. 5G provides the names of the C. glabrata strains of a portion of the rows of FIG. 5A (the names of the strains of the top and bottom columns are shown in FIG. 5A).

FIG. 6 is a heat map comprising shaded squares that illustrate the degree of inhibition of S. cerevisiae strains by yeasts expressing killer toxins. The intensity of the shading represents a qualitative assessment of the degree of growth inhibition and methylene blue staining by yeasts that express killer toxins. Darker gray shading represents a greater degree of growth inhibition. Light gray squares indicate no observable growth inhibition.

FIG. 7 is a heat map comprising shaded squares that illustrate the degree of inhibition of 11 Saccharomycetaceae yeast strains (C. castellii, N. bacillisporus, C. glabrata, N. delphensis, K. transvaalensis, K. spencerorum, K. lodderae, K. bulderi, T. delbrueckii, C. humilis, and T. phaffi) by various yeasts that express killer toxins. The intensity of the shading represents a qualitative assessment of the degree of growth inhibition and methylene blue staining by yeasts expressing killer toxins. Darker gray shading represents a greater degree of growth inhibition. Light gray squares indicate no observable growth inhibition. C. glabrata growth was inhibited by the greatest number of strains of yeasts that express killer toxins, as compared to other closely related yeast species.

FIG. 8 is a digital image illustrating cellulose chromatography results that demonstrate that dsRNAs of varying molecular weight are present in most killer toxin-expressing yeasts with activity against C. glabrata. All 9 killer toxin-expressing yeast strains tested (BJH001, EC1118, YO1620, YO1621, YO1622, YJM1307, YJM1290, YJM1341, and CYC1172) comprise dsRNAs that correspond to the molecular weight associated with totiviruses and satellite dsRNAs.

FIG. 9 is a graph of percentage of maximum kill zone size versus pH, illustrating killer toxin activity at various pH levels. Killer toxin activity was measured on agar plates at pH 4 to pH 5.5. Killer toxins expressed by all S. cerevisiae strains shown (with the exception of toxins expressed by strain SK1) showed activity at an acidic pH of 4.6. Strain SK1 showed highest toxin activity at pH 4.0, with activity declining as pH increased.

FIG. 10 is a digital image of an agar plate illustrating that purified K1 toxin is active against C. glabrata (ATCC2001) at 25° C. and 37° C. in synthetic vaginal-simulative growth medium and in YPD medium in a hypoxic atmosphere. The K1 toxin was purified using either ethanol precipitation or ammonium sulfate precipitation from a K1 toxin-expressing yeast strain (YJM1307). The purified toxin was spotted onto agar plates containing synthetic vaginal-simulative growth medium or YPD medium at different temperatures. The plates had been inoculated with C. glabrata (ATCC2001) prior to spotting of the purified toxin. The central zone of growth inhibition is large on both media types at 25° C., indicating active K1 toxin under each condition. The K1 toxin is also active at 37° C., but less so, as demonstrated by smaller zones of inhibition.

FIG. 11 is a digital image of an agar plate illustrating biological activity of the K1 and K2 toxins against the BY4741 yeast strain following separation using either ethanol or ammonium sulfate. K1 and K2 toxins were separated from yeast cells and cultures using either ethanol or ammonium sulfate and were plated separately on a BY4741 S. cerevisiae lawn. Separation using ammonium sulfate resulted in active K1 and K2 toxins that separately inhibited BY4741 growth. Ethanol precipitation is an effective alternative to ammonium sulfate for the purification of active K1 toxin (ethanol precipitated K1 toxin inhibited BY4741 growth) but is not suitable for the purification of active K2 (ethanol precipitated K2 toxin did not inhibit BY4741 growth). The K1 toxin was plated in the top two spots on the pictured plate and K2 toxin was plated in the bottom two spots. The left side of the plate contains toxins separated using ethanol precipitation and the right side of the plate contains toxins separated using ammonium sulfate. Dashed lines are added for ease of viewing the four spotted areas of the plate.

FIG. 12 is a digital image of an agar plate illustrating the stability of killer toxins at human body temperatures. K1 and K2 toxins were first precipitated with supersaturated ammonium sulfate and resuspended in 1× (10 g/L), 2× (20 g/L), 4× (40 g/L), 6× (60 g/L), 8× (80 g/L), or 10× (100 g/L) pH 4.6 yeast extract. Suspensions were incubated at 37° C. for 1 hour and 45 minutes. Five microliters were removed and plated to test for toxin activity every 15 minutes. The test lawn strain was S. cerevisiae BY4741 on pH 4.6 YPD stained with methylene blue. The K2 toxin maintained killing ability through all time points. The K1 toxin showed activity at the 0 and 15 minute time points, but not at the 30, 45, 60, 75, 90, or 105 minute time points. Note that the 30 minute time points were inadvertently reversed (these time points are labeled “switch” in the Figure), with the 30 minute time point for the K1 toxin placed within the K2 toxin test plate, and the 30 minute time point for the K2 toxin placed within the K1 toxin test plate.

FIG. 13A is a table listing 135 clinical and environmental C. glabrata isolates obtained from various sources (Example 1). Strain name (column 2), collection information (column 3), isolation site (column 3), and geographical origin (column 4) are provided for each strain.

FIG. 13B is a continuation of the table of FIG. 13A.

FIG. 13C is a continuation of the table of FIGS. 13A-B.

FIG. 13D is a continuation of the table of FIGS. 13A-C.

FIG. 14A lists 78 exemplary strains of killer toxin-producing yeasts. Yeast strain name (column 2), killer toxin expressed (if known; column 3), collection information (column 4), and origin (column 5) are provided for each strain.

FIG. 14B is a continuation of the table of FIG. 14A.

FIG. 14C is a continuation of the table of FIGS. 14A-B.

FIG. 14D is a continuation of the table of FIGS. 14A-C.

FIG. 15A lists 63 exemplary strains of killer toxin-producing yeasts that produce toxins that can inhibit C. glabrata. Yeast strain name (column 2), killer toxin expressed (if known; column 3), collection information (column 4), and origin (column 5) are provided for each strain.

FIG. 15B is a continuation of the table of FIG. 15A.

FIG. 15C is a continuation of the table of FIGS. 15A-B.

FIG. 16 is a digital image of an agar plate illustrating the effect of three different concentrations of yeast extract (0.5%, 1%, and 2%) on stabilization of K1 toxin in CM with 2% dextrose. S. cerevisiae YJM1307 was grown in complete liquid media (CM) with different concentrations of yeast extract. K1 toxin was precipitated from CM used for yeast growth, and was then used to challenge C. glabrata (ATCC2001) grown on YPD agar media (pH 4.6 with methylene blue).

FIG. 17 is a digital image of an agar plate depicting stabilization of K1 toxin at 37° C. using mannose, trehalose, or sucrose, as compared to K1 toxin suspended in a simple buffer. Precipitated K1 was incubated at 37° C. for up to 24 hours in sodium citrate buffer with or without three different carbohydrates. C. glabrata grown on YPD agar media (pH 4.6 with methylene blue) was challenged with toxin preparations and growth inhibition was indicated by the absence of growth.

FIG. 18 is a digital image of an agar plate showing the activity of K1 toxin extracted from the same S. cerevisiae culture once per day for three consecutive days. C. glabrata grown on YPD agar media (pH 4.6 with methylene blue) was challenged with the toxin preparations and growth inhibition was indicated by the absence of growth.

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Dec. 17, 2021, 86,016 bytes, which is incorporated by reference herein. In the accompanying sequence listing:

SEQ ID NO: 1 is an amino acid sequence of a K1 protein identified in S. cerevisiae strain YO-1490.

SEQ ID NO: 2 is an amino acid sequence of a K1 protein identified in S. cerevisiae strain YO-1482.

SEQ ID NO: 3 is an amino acid sequence of a K1 protein identified in S. cerevisiae strain YJM1307.

SEQ ID NO: 4 is an amino acid sequence of a K1 protein identified in S. cerevisiae strain YJM1287.

SEQ ID NO: 5 is an amino acid sequence of a K1 protein identified in S. cerevisiae strain YJM1077.

SEQ ID NO: 6 is an amino acid sequence of a K1 protein identified in S. cerevisiae strain YJM1290.

SEQ ID NO: 7 is an amino acid sequence of a K1 protein identified in S. cerevisiae strain Y-2429.

SEQ ID NO: 8 is an amino acid sequence of a K1 protein identified in S. cerevisiae strain YO-1621.

SEQ ID NO: 9 is an amino acid sequence of a K1 protein identified in S. cerevisiae strain YO-1622.

SEQ ID NO: 10 is an amino acid sequence of a K1 protein identified in S. cerevisiae strain BJH001.

SEQ ID NO: 11 is an amino acid sequence of a K1 protein identified in S. cerevisiae strain SK1.

SEQ ID NO: 12 is an amino acid sequence of a canonical K1 protein identified in S. cerevisiae.

SEQ ID NO: 13 is an amino acid sequence of a K2 protein identified in S. cerevisiae strain CYC1058.

SEQ ID NO: 14 is an amino acid sequence of a K2 protein identified in S. cerevisiae strain CYC1170.

SEQ ID NO: 15 is an amino acid sequence of a K2 protein identified in S. cerevisiae strain EC1118.

SEQ ID NO: 16 is an amino acid sequence of a K2 protein identified in S. cerevisiae strain NCYC1172.

SEQ ID NO: 17 is an amino acid sequence of a K2 protein identified in S. cerevisiae strain YJM1341.

SEQ ID NO: 18 is an amino acid sequence of a canonical K2 protein identified in S. cerevisiae.

SEQ ID NO: 19 is a nucleotide sequence of a K1 gene identified in S. cerevisiae strain YO-1490.

SEQ ID NO: 20 is a nucleotide sequence of a K1 gene identified in S. cerevisiae strain YO-1482.

SEQ ID NO: 21 is a nucleotide sequence of a K1 gene identified in S. cerevisiae strain YJM1307.

SEQ ID NO: 22 is a nucleotide sequence of a K1 gene identified in S. cerevisiae strain YJM1287.

SEQ ID NO: 23 is a nucleotide sequence of a K1 gene identified in S. cerevisiae strain YJM1077.

SEQ ID NO: 24 is a nucleotide sequence of a K1 gene identified in S. cerevisiae strain YJM1290.

SEQ ID NO: 25 is a nucleotide sequence of a K1 gene identified in S. cerevisiae strain Y-2429.

SEQ ID NO: 26 is a nucleotide sequence of a K1 gene identified in S. cerevisiae strain YO1621.

SEQ ID NO: 27 is a nucleotide sequence of a K1 gene identified in S. cerevisiae strain YO1622.

SEQ ID NO: 28 is a nucleotide sequence of a K1 gene identified in S. cerevisiae strain BJH001.

SEQ ID NO: 29 is a nucleotide sequence of a K1 gene identified in S. cerevisiae strain SK1.

SEQ ID NO: 30 is a nucleotide sequence of a canonical K1 gene identified in S. cerevisiae.

SEQ ID NO: 31 is a nucleotide sequence of a K2 gene identified in S. cerevisiae strain CYC1058.

SEQ ID NO: 32 is a nucleotide sequence of a K2 gene identified in S. cerevisiae strain CYC1170.

SEQ ID NO: 33 is a nucleotide sequence of a K2 gene identified in S. cerevisiae strain EC1118.

SEQ ID NO: 34 is a nucleotide sequence of a K2 gene identified in S. cerevisiae strain NCYC1172.

SEQ ID NO: 35 is a nucleotide sequence of a K2 gene identified in S. cerevisiae strain YJM1341.

SEQ ID NO: 36 is a nucleotide sequence of a canonical K2 gene identified in S. cerevisiae.

SEQ ID NO: 37 is a nucleotide sequence of a K1L gene (GENBANK® Accession No. MW248137.1) identified in S. paradoxus.

SEQ ID NO: 38 is an amino acid sequence of a K1L protein (GENBANK® Accession No. QQX23408.1) identified in S. paradoxus.

### DETAILED DESCRIPTION

### I. Terms and Definitions

Unless otherwise noted, technical terms are used according to conventional usage as would be understood by a person of ordinary skill in the art. Definitions of common terms in molecular biology may be found in Lewin's Genes X, ed. Krebs et al, Jones and Bartlett Publishers, 2009 (ISBN 0763766321); Kendrew et al. (eds.), The Encyclopedia of Molecular Biology, Blackwell Publishers, 1994 (ISBN 0632021829); Robert A. Meyers (ed.), Molecular Biology and Biotechnology: A Comprehensive Desk Reference, Wiley, John & Sons, Inc., 1995 (ISBN 0471186341); and George P. Rédei, Encyclopedic Dictionary of Genetics, Genomics, Proteomics and Informatics, 3rd Edition, Springer, 2008 (ISBN: 1402067534).

The following explanations of terms and abbreviations are provided to better describe the present disclosure and to guide those of ordinary skill in the art in the practice of the present disclosure. As used herein, “comprising” means “including” and the singular forms “a” or “an” or “the” refer to one or more than one unless the context clearly dictates otherwise. The term “or” refers to a single element of stated alternative elements or a combination of two or more elements, unless the context clearly indicates otherwise.

All publications, patent applications, patents, and other references mentioned herein are incorporated by reference in their entirety for all purposes. All sequences associated with GenBank Accession Nos. mentioned herein are incorporated by reference in their entirety as of the present application's priority date. In case of conflict, the present specification, including explanations of terms, will control.

Although methods and materials similar or equivalent to those described herein can be used to practice or test the present disclosure, suitable methods and materials are described below. The materials, methods, and examples are illustrative only and not intended to be limiting. Other features of the disclosure will be apparent to a person of ordinary skill in the art from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Amino acid residues in the disclosed sequence listing may be conservatively substituted or replaced by another residue with similar properties and characteristics. Typically, conservative substitutions have little to no impact on the activity of a resulting peptide. In one non-limiting example, a tyrosine reside in one peptide of a composition is substituted with a tryptophan residue. A peptide can be produced by chemical substitution to include one or more conservative amino acid substitutions, or can be produced by manipulating the nucleic acid sequence that encodes that peptide using, for example, standard procedures such as PCR or site-directed mutagenesis. Table 1 below provides conservative amino acid substitutions for expressly disclosed peptide sequences that are within the scope of the present disclosure.

TABLE 1
Conservative Amino Acid Substitutions

| Definition | Amino Acid | Symbol |
|---|---|---|
| Amino acids with aliphatic R-groups | Glycine | Gly-G |
| | Alanine | Ala-A |
| | Valine | Val-V |
| | Leucine | Leu-L |
| | Isoleucine | Ile-I |
| Amino acids with hydroxyl R-groups | Serine | Ser-S |
| | Threonine | Thr-T |
| Amino acids with sulfur-containing R-groups | Cysteine | Cys-C |
| | Methionine | Met-M |
| Acidic amino acids | Aspartic Acid | Asp-D |
| | Asparagine | Asn-N |
| | Glutamic Acid | Glu-E |
| | Glutamine | Gln-Q |
| Basic amino acids | Arginine | Arg-R |
| | Lysine | Lys-K |
| | Histidine | His-H |
| Amino acids with aromatic rings | Phenylalanine | Phe-F |
| | Tyrosine | Tyr-Y |
| | Tryptophan | Trp-W |
| Imino acids | Proline | Pro-P |

To facilitate review of the various embodiments of this disclosure, the following explanations of specific terms are provided:

Administer, Administering, Administration: As used herein, administering a composition (e.g. a therapeutic composition) to a subject means to apply, give, or bring the composition into contact with the subject. Compositions may be administered to a subject by, for example, the subject, a healthcare professional, or another individual. Exemplary routes of administration include, but are not limited to, parenteral, intramuscular, and/or subcutaneous injection, oral administration, topical administration, and/or vulvovaginal administration, such as intravaginal administration and/or vulvar administration, such as, for example, for treating or preventing an infection, such as a yeast infection (such as VVC), such as a yeast infection caused by a yeast of genus Candida, such as a yeast infection caused by Candida glabrata.

Administration may comprise providing, such as providing to a subject, one or more compositions disclosed herein in the form of an ointment, cream, suppository, solid (such as a capsule, ovule, tablet, pill and/or suppository), solution, suspension, foam, film, gel, liposomal composition, or combinations thereof. Compositions administered herein may also be administered on or within, or both, an applicator, such as a tampon, vaginal ring, pillow, puff, sponge, osmotic pump system, an applicator useful for administering (such as topically and/or vulvovaginally) a cream and/or ointment, an applicator useful for administering (such as intravaginally) a capsule, ovule, tablet, pill, and/or suppository, or combinations thereof. In some embodiments, the compositions may be administered using one or more applicators. In other embodiments, no applicator is required for administration.

Combination: A combination includes two or more components that are administered such that the effective time period of at least one component overlaps with the effective time period of at least one other component. A component may be a composition. In some embodiments, the effective time periods of all components administered overlap with each other. In an exemplary embodiment of a combination comprising three components, the effective time period of the first component administered may overlap with the effective time periods of the second and third components, but the effective time period of the second component independently may or may not overlap with that of the third component. A combination may be a composition comprising the components, a composition comprising two or more individual components, or a composition comprising one or more components and another separate component (or components) or composition(s) comprising the remaining component(s). In some embodiments, the two or more components may comprise two or more different components administered substantially simultaneously or sequentially in any order, the same component administered at two or more different times, or a combination thereof.

Effective amount: The term “effective amount” refers to the amount of an agent (such as one or more embodiments provided herein alone, in combination, or potentially in combination with other therapeutic agent(s)) that is sufficient to induce a desired biological result. That result may be amelioration or alleviation of the signs, symptoms, or causes of a disease, or any other desired alteration of a biological system. The amount can vary with the condition being treated, the severity and/or stage of advancement of the condition, the age of the patient to be treated, the type and concentration of formulation applied, and other factors known in the art. In some embodiments, an effective amount of a therapeutic composition is an amount which, when administered to a subject, is sufficient to alleviate the symptoms of infection by a pathogenic organism of interest. Such a response may comprise, for instance, a reduction in the number and/or concentration of such pathogenic organisms, such as a particular yeast species or strain, on or within the subject, for example at the location of the infection. Appropriate amounts in any given instance will be readily apparent to those of ordinary skill in the art or capable of determination by routine experimentation such as treatment of a subject and observation of the infection and/or one or more symptoms of the infection in the subject.

Expression Control Sequences: “Expression control sequences” are nucleic acid sequences that regulate the expression of a heterologous nucleic acid sequence to which they are operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression control sequences can also include one or more selectable marker genes (such as an antibiotic resistance gene, and/or a fluorescent, luminescent, or colorimetric marker). Expression control sequences can also include regulatory sequences that allow for controlled modulation (such as inducement, prevention, increase, and/or decrease) of transcription and translation of an inserted gene or genes. Such sequences allow for controlled expression of a gene of interest, such as by a molecular switch. Exemplary inducible promoters that can be used with the disclosed embodiments include an inducible galactose promoter and/or a constitutive GPD promoter. Markers used to maintain expression plasmids can include auxotrophic markers, such as URA3, TRP1, and/or ADE2, that can complement nutritional deficits in a yeast genome.

Expression of a killer toxin can in some embodiments serve as a selectable marker. In some embodiments, a cell wherein killer toxin expression is lost may die due to lack of expression of a killer toxin antidote. In some embodiments, killer toxin expression stability may be optimized by integrating one or more killer toxin genes into the yeast genome.

Expression vector: An “expression vector” is a vector comprising a recombinant polynucleotide comprising expression control sequences operatively linked to a nucleotide sequence to be expressed. An expression vector comprises sufficient elements for expression; other elements for expression can be supplied, for example, by a host cell and/or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments expression vectors are cosmids and plasmids.

Host cell: “Host cell” refers to a cell or cells in which a vector can be propagated and its DNA expressed. The cell can be eukaryotic or prokaryotic. The cell can be mammalian, such as a swine cell. “Host cell” also includes any progeny of the subject host cell. It is understood that all progeny may or may not be identical to the parental cell since mutations may occur during replication. Such progeny are understood to be included when the term “host cell” is used.

Infection: Infection or challenge means that the subject has been exposed to organisms that may produce disease causing the subject to suffer one or more clinical signs of the diseases when they have been exposed to such organisms.

Isolated: To be significantly separated from other agents. An “isolated” biological component (such as a nucleic acid molecule, protein, or cell) has been substantially separated, produced apart from, or purified away from other biological components in the cell of the organism in which the component occurs, for example, other chromosomal and extra-chromosomal DNA and RNA, and proteins. Nucleic acid molecules and proteins which have been “isolated” also include nucleic acid molecules and proteins purified by standard purification methods, such as ethanol precipitation and purification using ammonium sulfate. Additional isolation and/or purification methods known in the art include, but are not limited to, size-exclusion chromatography, affinity chromatography, cellulose affinity chromatography, ultracentrifugation, ultrafiltration, and centrifugal partition chromatography. The term “isolated” also embraces nucleic acid molecules and proteins prepared by recombinant expression in a host cell as well as chemically synthesized proteins and nucleic acids. Samples of isolated biological components include samples of the biological component wherein the biological component represents greater than 90% (for example, greater than 95%, such as greater than 98%) of the sample.

An “isolated” microorganism (such as isolated Saccharomyces cerevisiae strain BY4741) has been substantially separated or purified away from microorganisms of different types, strains, or species. In some embodiments, the microorganism is isolated as a population of genetically identical clones. Microorganisms can be isolated by a variety of techniques, including serial dilution and culturing and resistance to certain chemicals, such as antibiotics. In some examples, an isolated strain of Saccharomyces is at least 70% (for example, at least 75%, at least 85%, at least 95%, at least 98%, at least 99%, at least 99.99%, or substantially 100%) pure. In some embodiments, the isolated strain is at least 99%, at least 99.99%, or substantially 100% pure.

Killer toxin: A “killer toxin” or “toxin” as disclosed herein is a protein that may have biological activity against one or more organisms, such as one or more fungi. In particular embodiments, the fungus is a yeast. Such biological activity may comprise, but is not limited to, cell growth inhibitory activity and/or activity that directly or indirectly causes cell death, such as against one or more pathogenic organisms.

Killer toxins are expressed by organisms and a person of ordinary skill of the art understands that, unless otherwise specified, killer toxins, as used herein, are understood to be separated from the organisms or cellular fragments thereof. Additionally, a killer toxin may be purified, such as by protein purification techniques known to persons of ordinary skill in the art, and may have a purity of at least 50%, at least 60%, at least 70%, at least 80%, at least 90%, at least 95%, at least 96%, at least 97%, at least 98%, at least 99%, or substantially 100%.

A killer toxin may be expressed endogenously by one or more yeasts, such as a yeast of genus Saccharomyces. Killer toxins may also be expressed exogenously by an organism transformed to express one or more killer toxins, such as a recombinant yeast cell, a recombinant bacterial cell, a recombinant virus, a recombinant animal cell, or a transgenic animal Additionally, a yeast that endogenously expresses one or more killer toxins may be transformed and/or otherwise genetically manipulated to express one or more different toxins and/or greater amounts of the same toxin.

A killer toxin may, but need not completely, kill or inhibit the growth of a susceptible organism. A killer toxin may inhibit the growth of a susceptible organism to some degree, such as at least 20%, at least 30%, at least 40%, at least 50%, at least 60%, at least 70%, at least 75%, at least 80%, at least 85%, at least 90%, at least 95%, at least 99%, or substantially 100%. In one non-limiting example, a killer toxin expressed by a Saccharomyces cerevisiae yeast may inhibit the growth of a Candida yeast, such as Candida glabrata, by at least 80%, by at least 90%, by at least 95%, by at least 99%, or substantially 100%.

A “killer toxin-expressing organism” or an “organism that expresses a killer toxin” or an “organism expressing a killer toxin” comprises one or more killer toxins. Such an organism may express one or more killer toxins, such as endogenously, or may be transformed to express one or more killer toxins, such as a recombinant organism. Exemplary killer toxin-expressing organisms can include, but are not limited to, a yeast, a recombinant yeast, a recombinant bacterial cell, a recombinant animal cell, a recombinant virus, and a transgenic animal. Additionally, a killer toxin-expressing organism that endogenously expresses one or more killer toxins may also be transformed and/or otherwise genetically manipulated to express one or more killer toxins, such as one or more additional copies of an endogenously expressed killer toxin, and/or a killer toxin different from a killer toxin expressed endogenously by the organism.

Pharmaceutically acceptable excipient: A pharmaceutically acceptable excipient of any embodiment disclosed herein is a substance, other than the active ingredient or ingredients, such as a killer toxin and/or an organism that expresses a killer toxin, that is included in a formulation of the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, dextrose, yeast extract, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is an excipient that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, yeast extract, dextrose, aluminum hydroxide, alum, aluminum phosphate, killed bacteria, squalene, detergents, cytokines, paraffin oil, and combination adjuvants, such as Freund's complete adjuvant or Freund's incomplete adjuvant.

“Pharmaceutically acceptable carrier” refers to an excipient that is a carrier or vehicle, such as a suspension aid, solubilizing aid, or aerosolization aid. Remington: The Science and Practice of Pharmacy, The University of the Sciences in Philadelphia, Editor, Lippincott, Williams, & Wilkins, Philadelphia, Pa., 21^{st} Edition (2005), incorporated herein by reference, describes exemplary compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise components that can be administered topically and/or to a mucosal surface, and that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by oral, topical, parenteral, intramuscular, and/or subcutaneous injection). In addition to biologically-neutral carriers, pharmaceutical compositions to be administered can contain minor amounts of non-toxic auxiliary substances, such as, but not limited to, wetting or emulsifying agents, preservatives, and pH buffering agents, for example sodium acetate or sorbitan monolaurate.

“Pharmaceutically acceptable salt” refers to an excipient that is a pharmaceutically acceptable salt of a compound that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

“Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.) In particular disclosed embodiments, amide compounds may be a formate, trifluoroactate, hydrochloride or sodium salt.

Preventing, treating, or ameliorating a disease: “Preventing” a disease refers to inhibiting the development of a disease. “Treating” refers to a therapeutic intervention that ameliorates a sign or symptom of a disease or pathological condition after it has begun to develop. “Ameliorating” refers to the reduction in the number or severity of one or more signs or symptoms of a disease.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In some examples, a promoter is bi-directional. Native and non-native promoters can be used to drive expression of a gene, such as K1 and/or K2. Exemplary promoters that can be used include, but are not limited to, a galactose-inducible promoter GAL1-10, a copper inducible promoter from CUP1, a constitutive promoter GPD, and/or a constitutive promoter ADH1. A person of ordinary skill in the art understands how to use and include promoters. However, additional information concerning promoters can be found in Crabtree et al. (2019) Viruses 11(1):70; Gier et al. (2017) Toxins 9(11):345; Giesselmann et al. (2017) Microbial Cell Factories 16(1):228; Dignard et al. (1991) Molecular & General Genetics 227(1):127; and Meškauskas et al. (1992) Gene 111(1):135, which are incorporated by reference herein in their entireties.

Other examples of promoters include, but are not limited to the SV40 promoter, the CMV enhancer-promoter, and the CMV enhancer/β-actin promoter. Both constitutive and inducible promoters can be used in the methods provided herein (see e.g., Bitter et al., Methods in Enzymology 153:516-544, 1987, which is incorporated herein by reference). Also included are those promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Purified: A purified protein, virus, nucleic acid, or other compound is one that is isolated in whole or in part from associated proteins and other contaminants. In certain embodiments, the term “substantially purified” refers to a protein, virus, nucleic acid, or other compound that has been isolated from a cell, cell culture medium, or other crude preparation and subjected to purification to remove various components of the initial preparation, such as proteins, cellular debris, and other components.

Recombinant: A recombinant nucleic acid molecule or protein is one that has a sequence that is not naturally occurring or has a sequence that is made by an artificial combination of two otherwise separated segments of sequence. In particular examples, this artificial combination is accomplished by chemical synthesis or by the artificial manipulation of isolated segments of nucleic acids, for example, by genetic engineering techniques such as those described in Sambrook et al. (ed.), Molecular Cloning: A Laboratory Manual, 3d ed., vol. 1-3, Cold Spring Harbor Laboratory Press, Cold Spring Harbor, N.Y., 2001. The term recombinant includes nucleic acid molecules that have been altered solely by addition, substitution, or deletion of a portion of the nucleic acid molecule. A recombinant or transformed organism or cell, such as a recombinant Saccharomyces, is one that includes at least one exogenous nucleic acid molecule, such as one used to genetically inactivate an endogenous K1 gene, and/or one used to express a non-native protein, such as exogenous K2 nucleic acid coding sequences.

Sequence identity/similarity: The identity/similarity between two or more nucleic acid sequences, or between two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. In some embodiments, one or more disclosed proteins may comprise one or more amino acid sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to an amino acid sequence or sequences of SEQ ID NOs. 1-18 or SEQ ID NO: 38. In some embodiments, one or more disclosed nucleic acid molecules encoding one or more proteins of SEQ ID NOs. 1-18 or SEQ ID NO: 38 may comprise one or more nucleotide sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to a corresponding one or more nucleotide sequences, such as a corresponding nucleotide sequence of any one of SEQ ID NOs. 19-37, encoding the one or more proteins.

Sequence alignment methods for comparison and to determine sequence identity or similarity are known to those of ordinary skill in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Subject: A “subject” is any multi-cellular vertebrate organism, a category that includes both human and non-human mammals (such as avians, mice, rats, rabbits, sheep, swine, horses, cows, and non-human primates). In certain embodiments, the subject is human.

Transformed: A cell, such as a fungal cell, into which a nucleic acid molecule has been introduced, for example using molecular biology methods. As used herein, the term transformation encompasses all techniques by which a nucleic acid molecule might be introduced into such a cell, including, but not limited to chemical methods (e.g., calcium-phosphate transfection), physical methods (e.g., electroporation, microinjection, particle bombardment, particle gun acceleration), fusion (e.g., liposomes), receptor-mediated endocytosis (e.g., DNA-protein complexes, viral envelope/capsid-DNA complexes), transfection with plasmid and/or viral vectors, and by infection by viruses such as recombinant viruses.

Vector: A vector is a nucleic acid molecule allowing insertion of foreign nucleic acid without disrupting the ability of the vector to replicate and/or integrate in a host cell. A vector can transduce, transform or infect a cell, thereby causing the cell to express nucleic acids and/or proteins other than those native to the cell. A vector can include nucleic acid sequences that permit it to replicate in a host cell, such as an origin of replication. An insertional vector is capable of inserting itself into a host nucleic acid. A vector can also include one or more selectable marker genes (such as an antibiotic resistance gene, and/or a fluorescent or colorimetric marker) and other genetic elements. An expression vector is a vector that contains regulatory sequences that allow transcription and translation of an inserted gene or genes. Regulatory sequences may allow for controlled expression of a gene of interest, such as by a molecular switch. A vector optionally includes materials to aid in achieving entry of the nucleic acid into the cell, such as a viral particle, liposome, protein coating or the like. In one example, a vector is a plasmid.

### II. Overview of Embodiments

“Killer toxins” are proteins that can inhibit the growth of organisms including yeasts and other fungi. Killer toxins can selectively inhibit the growth of certain fungal pathogens and are generally considered non-toxic to mammals. While different species and strains of yeasts express killer toxins that reportedly inhibit growth of certain C. glabrata strains under laboratory conditions, the killer toxins responsible for these antifungal activities have not been identified, nor has the effectiveness of these killer toxins, or of the yeasts that express these killer toxins, been tested against diverse C. glabrata clinical isolates, many of which are drug resistant.

Certain Saccharomyces yeasts comprise one or more double-stranded RNA (dsRNA) viruses that support the maintenance of satellite dsRNAs that encode “killer toxins.” Without being bound to a particular theory, totiviruses may be responsible for the replication of the satellite dsRNAs that encode killer toxins, such that a dsRNA satellite may not be able to replicate absent a totivirus. Killer toxin expression may further be reliant on host cell machineries for translation.

Killer toxin mRNA can be produced by the viral RNA-dependent RNA polymerase (RdRp) and can lack a 5′ cap that is essential for the initiation of translation. The virus capsid protein may be responsible (in concert with the activity of the RdRp) for acquiring a 5′ cap from a yeast mRNA by a cap-snatching mechanism. Killer toxin mRNA production may thus be dependent on viral proteins. Killer toxins can be expressed and excreted into the environment by yeasts and can inhibit the growth of and/or cause cell death in adjacent susceptible competitor yeasts (see Example 1). Currently, nine different dsRNA-encoded (K1, K2, K28, K1us, K74, K21, K62, K45, and K1L) and two yeast genome-encoded (KHR and KHS) killer toxins have been identified in Saccharomyces yeasts.

The present disclosure concerns compositions comprising killer toxins, organisms that express such toxins, and methods of using such compositions to treat and/or prevent yeast infections in mammals, including humans. For example, disclosed herein are data demonstrating for the first time that the important human pathogen Candida glabrata is wholly susceptible to canonical killer toxins encoded by dsRNAs found within certain S. cerevisiae (see Example 1) and/or certain S. paradoxus. That the disclosed killer toxins and/or related yeast strains may be used to treat yeast infections, such as, but not limited to, C. glabrata in animals, such as humans, represents a paradigm shift in the application of killer toxins and killer toxin-expressing yeasts as therapeutics.

Life-threatening bloodstream infections cause by yeasts, such as Candida yeasts, are relatively rare. However, vulvovaginal candidiasis (VVC) caused by yeasts is highly prevalent and effects a majority of women at least once in their lifetimes. Candida yeasts, such as C. glabrata, are thought to be a major cause of persistent and drug resistant VVC, and C. glabrata has been isolated from the vaginal mucosa of patients. Recurrent VVC causes an estimated worldwide economic burden in high income countries of $14 to $39 billion annually. Accordingly, Candida yeasts, such as C. glabrata, are of great clinical concern because of their ability to cause disease in vulnerable patients.

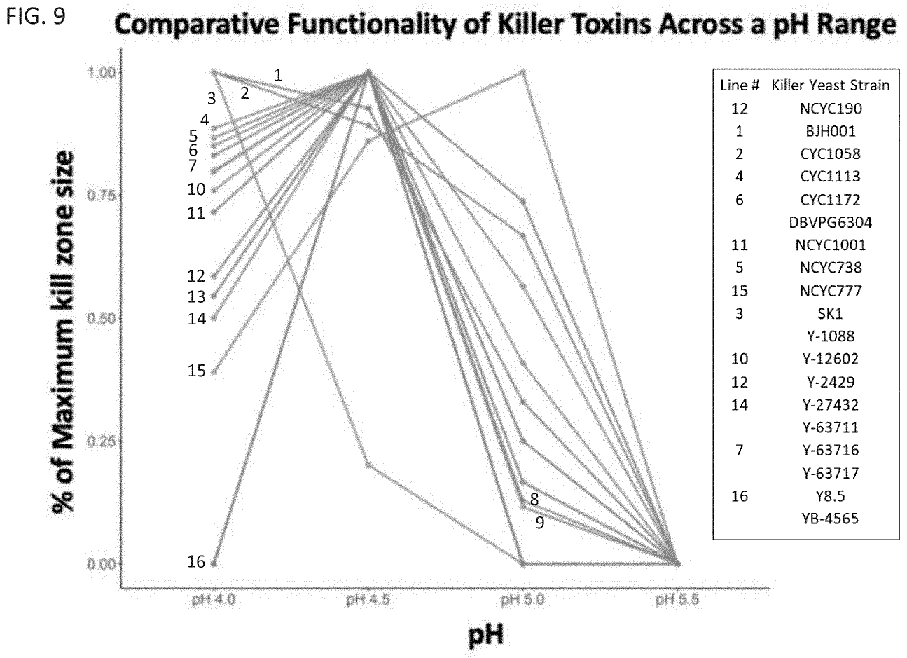

Killer toxins are useful for combatting VVC because of their activity against Candida yeasts, such as C. glabrata. Further, killer toxins, such as those identified herein, typically are active at an acidic pH of 4.6 (FIG. 9), which is about the pH of the vaginal mucosa. In contrast, commonly used topical therapeutics, such as Amphotericin B and azoles, are less effective at this acidic pH. The studies described herein show that when yeasts that express killer toxins are grown in vaginal-simulative media under hypoxic atmosphere conditions, there is no loss of killer toxin activity against C. glabrata (see Example 1).

Many strains of Saccharomyces yeasts comprise satellite double-stranded RNAs (dsRNAs) that encode killer toxins that have antifungal activity. Nine different dsRNA-encoded (K1, K2, K28, K1us, K74, K21, K62, K45, and K1L) and two genome-encoded (KHR and KHS) killer toxins have been identified in Saccharomyces yeasts. Organisms, such as certain yeasts, that express (such as endogenously and/or exogenously) killer toxins may inhibit the growth of pathogenic fungi relevant to human health and agriculture. However, previous studies suggested that individual killer toxins typically do not have activity across all strains of a given fungal species. However, surprisingly, the present inventors found that substantially all of the tested strains of Candida glabrata from multiple places around the world were susceptible to the disclosed killer toxins, particularly K1 and K2 toxins.

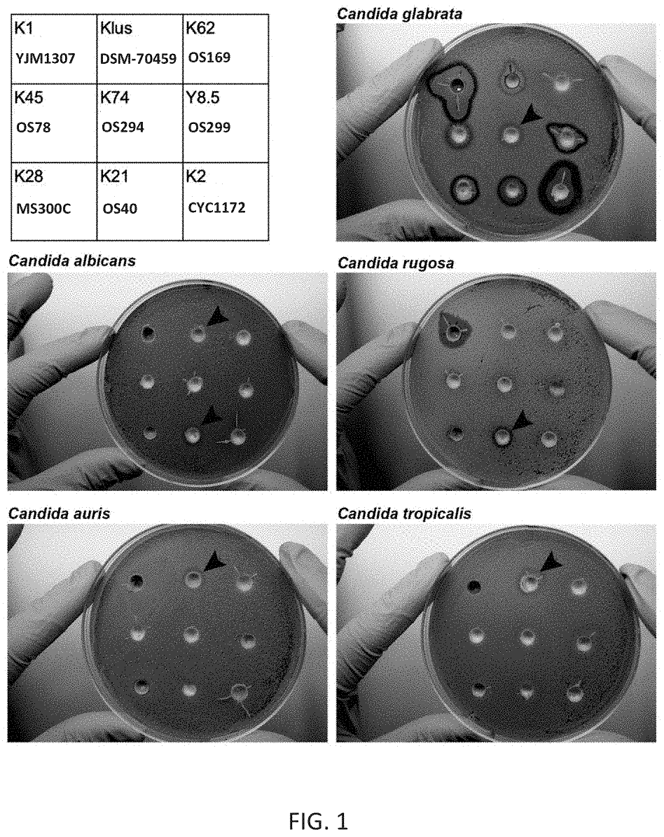

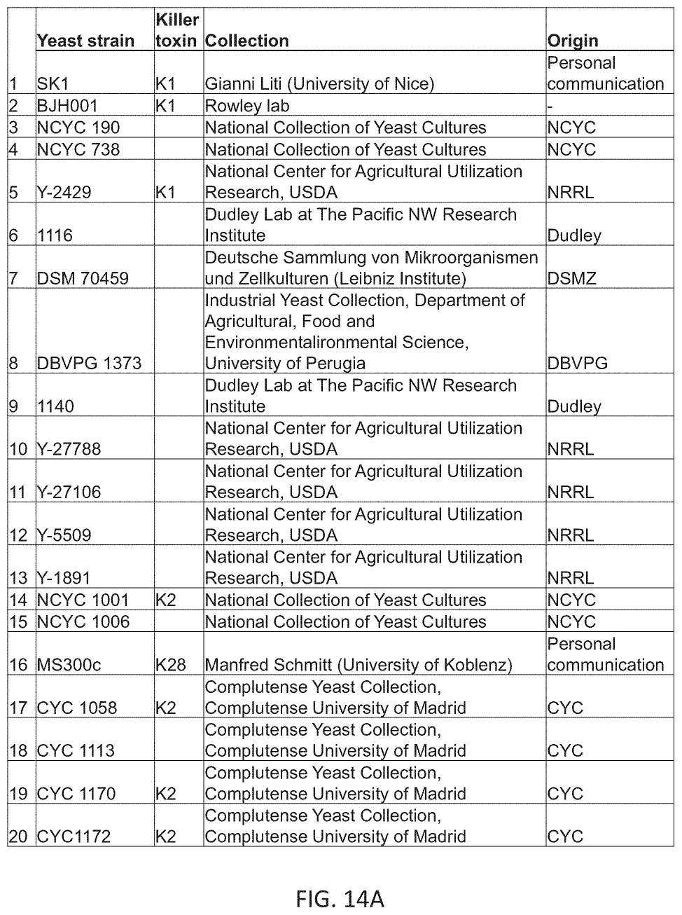

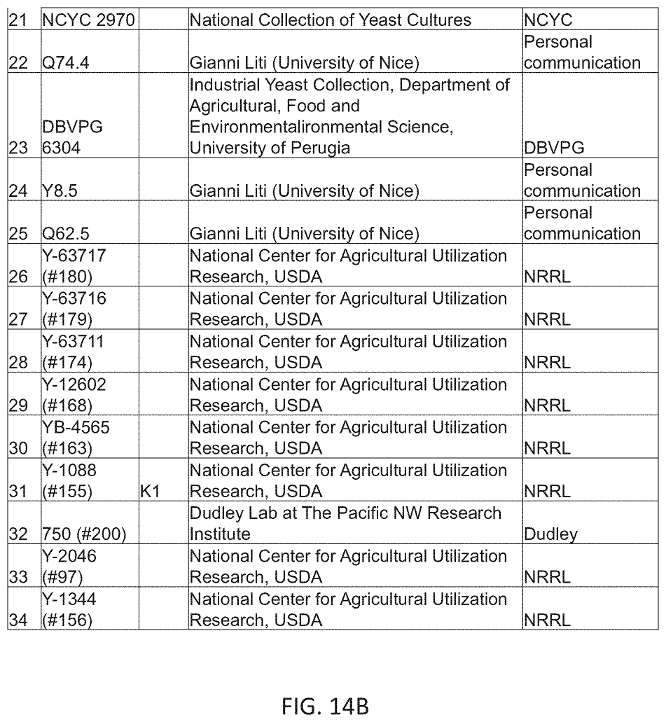

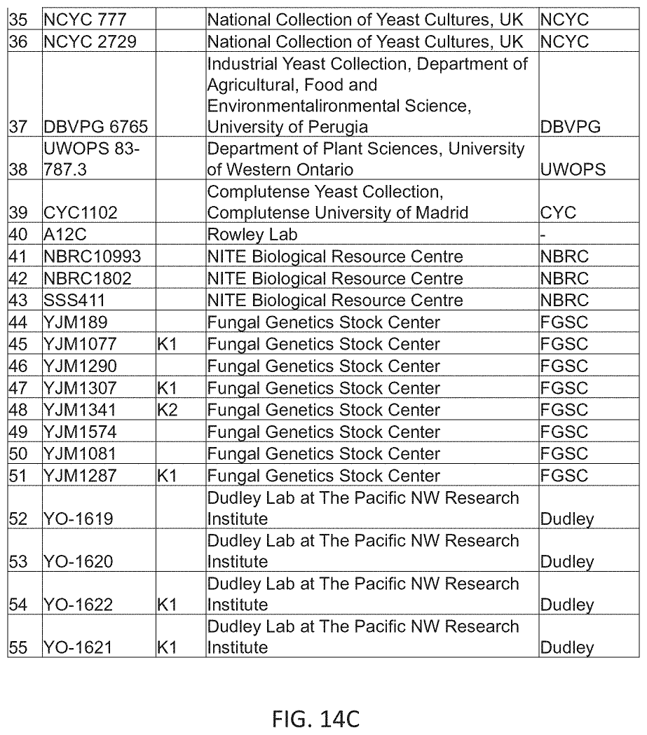

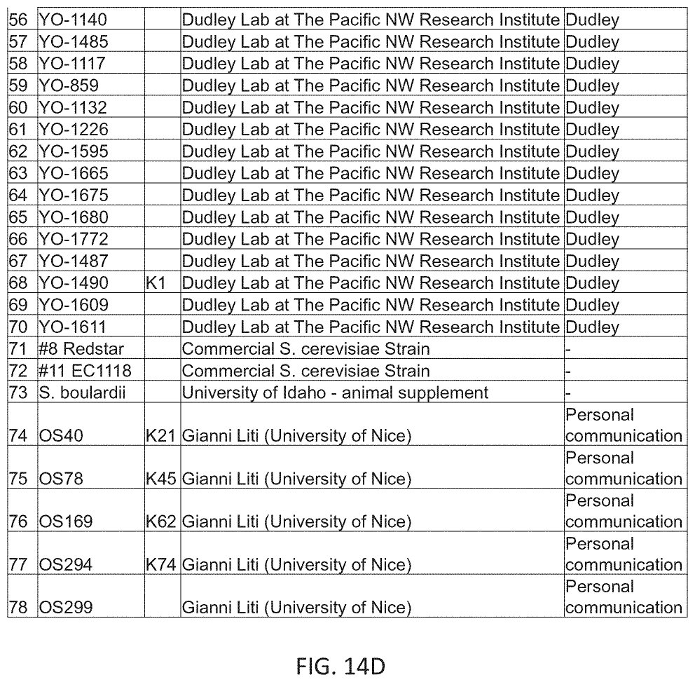

Saccharomyces yeast strains were screened for the expression of killer toxins using in vitro agar plate-based assays (Example 1). More than 76 yeasts were identified that were able to express killer toxins that inhibited growth of the yeast Saccharomyces cerevisiae (FIG. 14). Further in vitro assays revealed that a subset of these killer toxin-expressing yeasts were biologically active against human-pathogenic yeasts, including Candida yeasts, such as Candida glabrata, Candida rugosa, Candida auris, Candida albicans, and Candida tropicalis (FIG. 1). Of eight tested types of killer toxins expressed by the killer toxin-expressing yeast strains (including toxins K1, K2, K21, K28, K45, K1us, K74, and K62), all but K62 were able to inhibit the growth of the type strain of the pathogen C. glabrata (ATCC_2001) in vitro (FIG. 1). Further, the K1L toxin, which appears to share certain structural and functional similarities with the canonical K1 toxin, was recently identified as expressed in S. paradoxus.

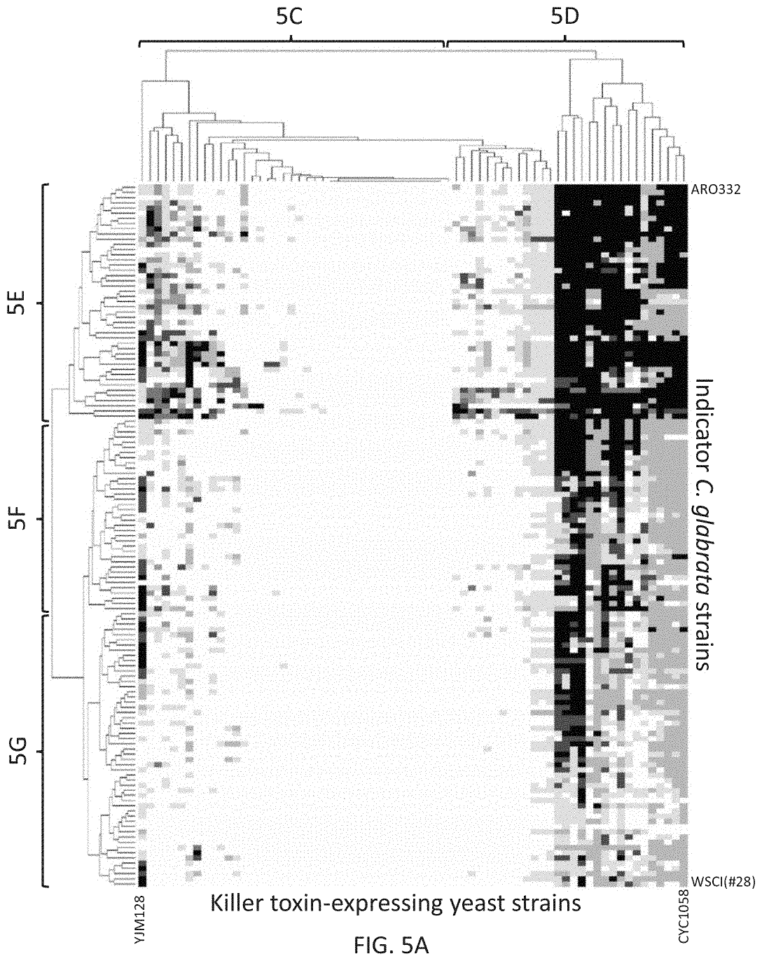

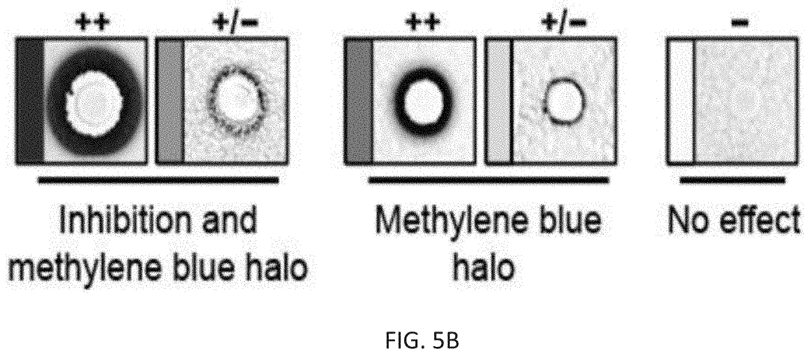

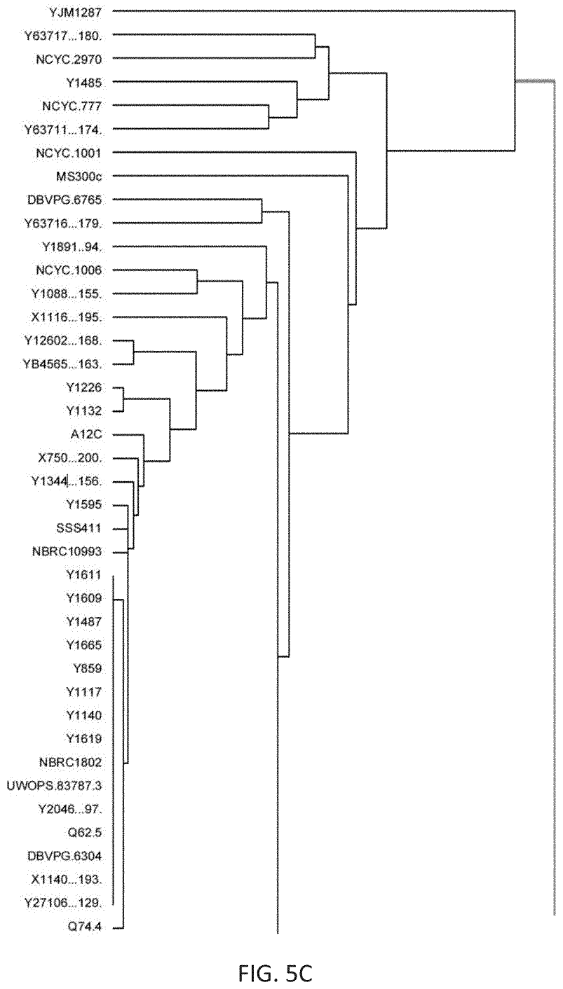

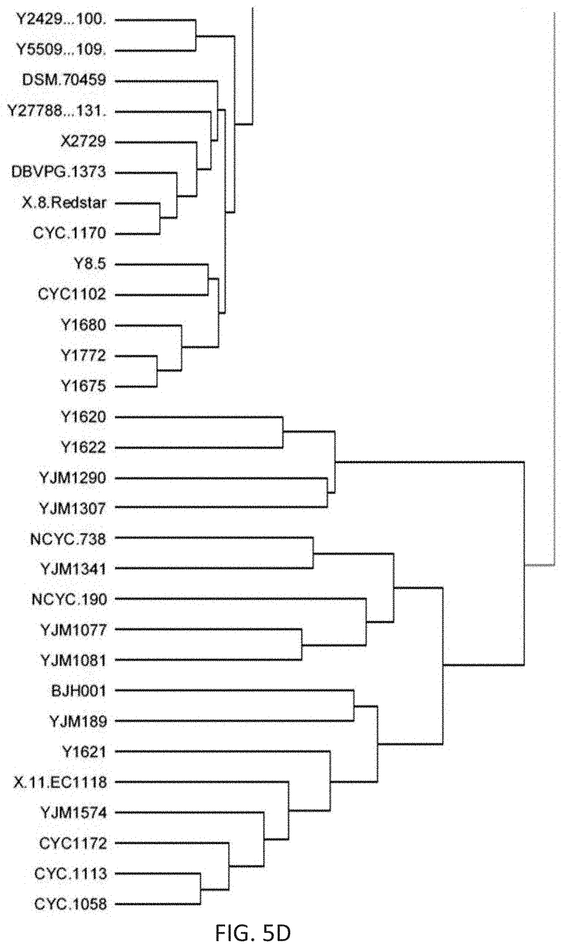

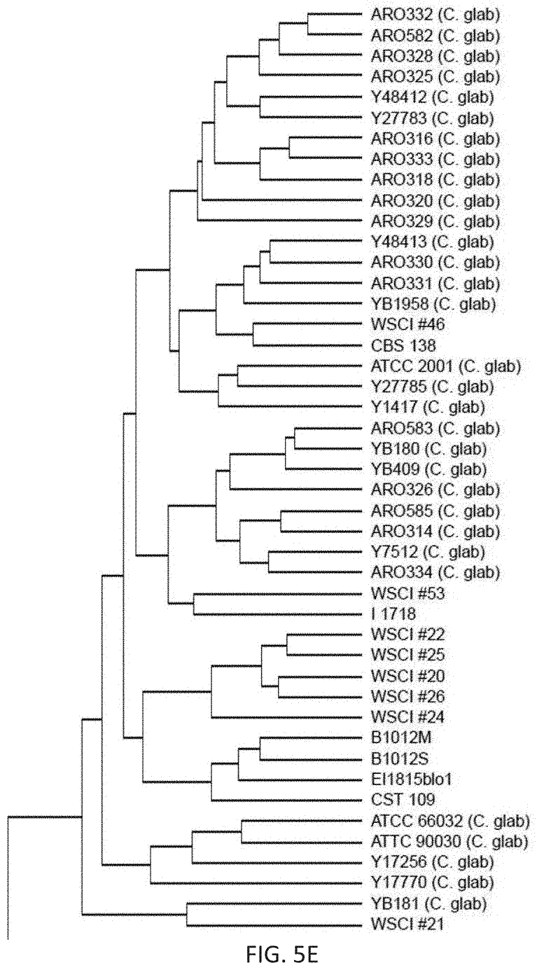

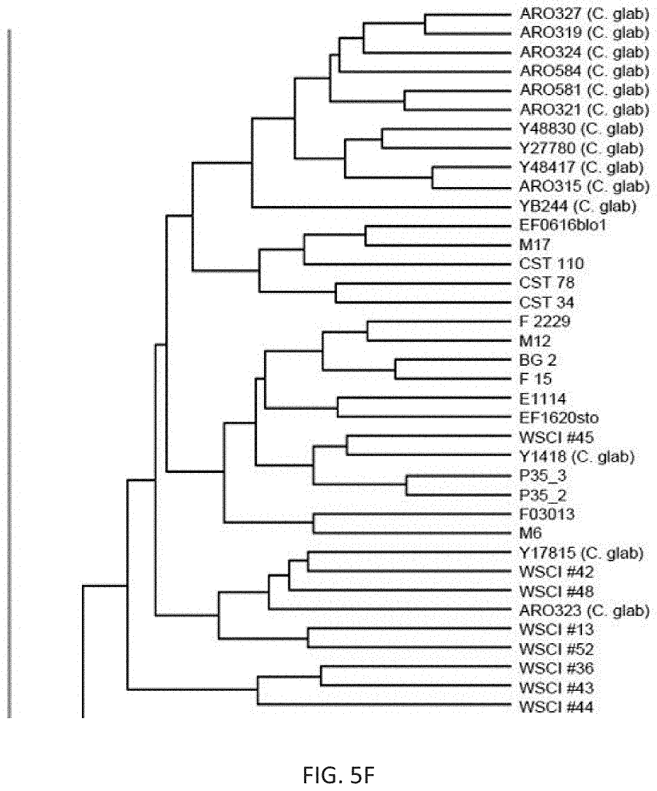

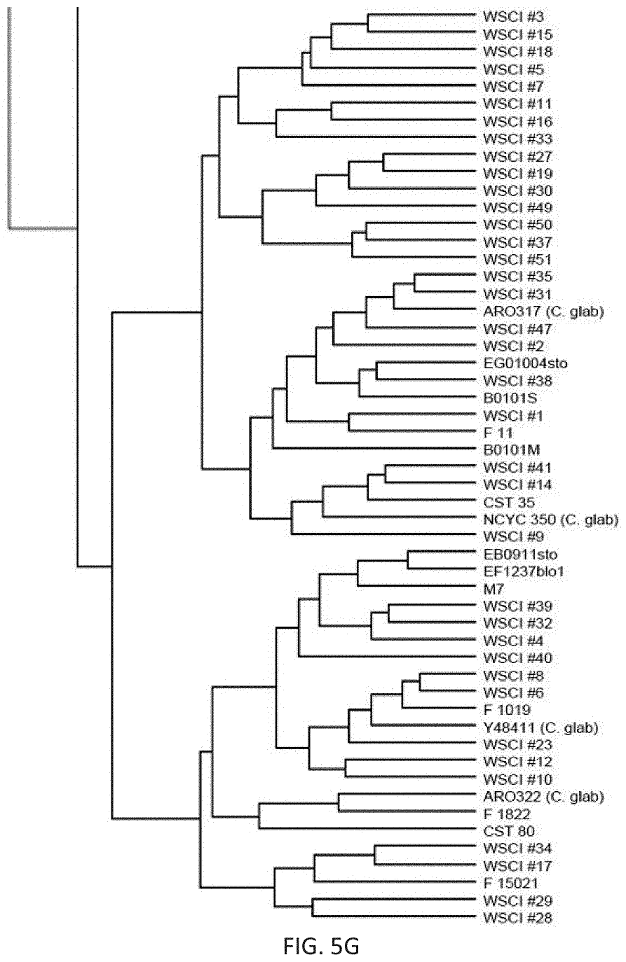

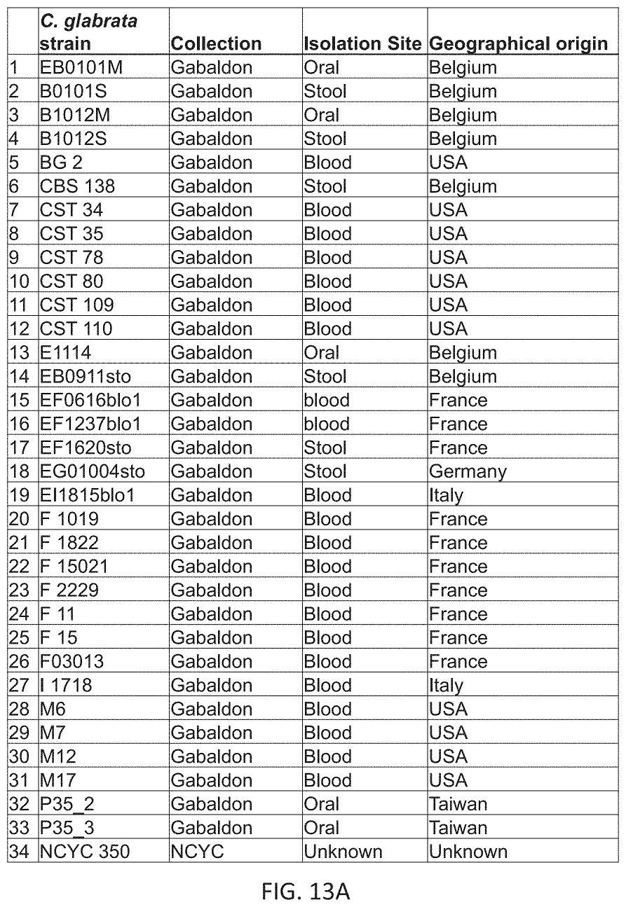

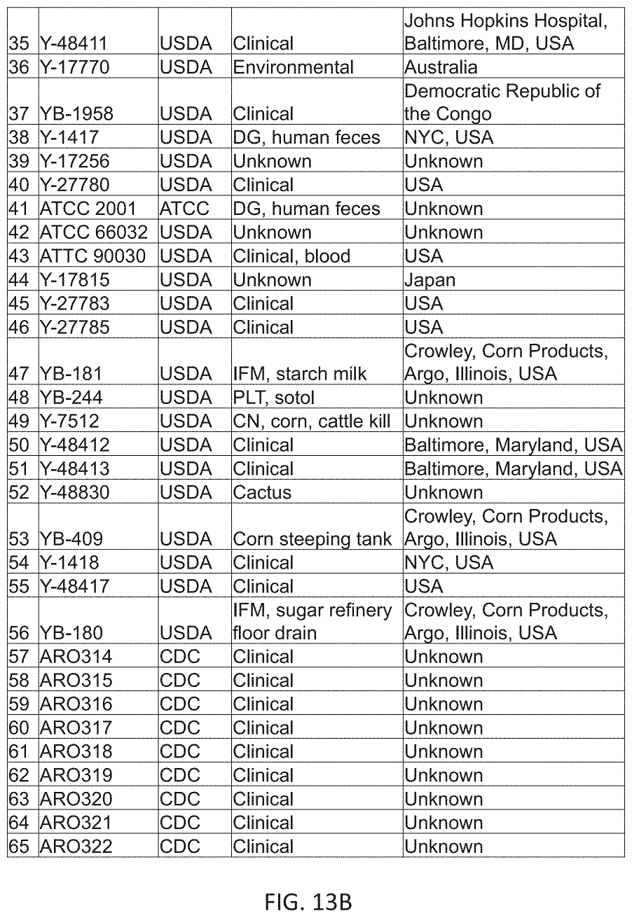

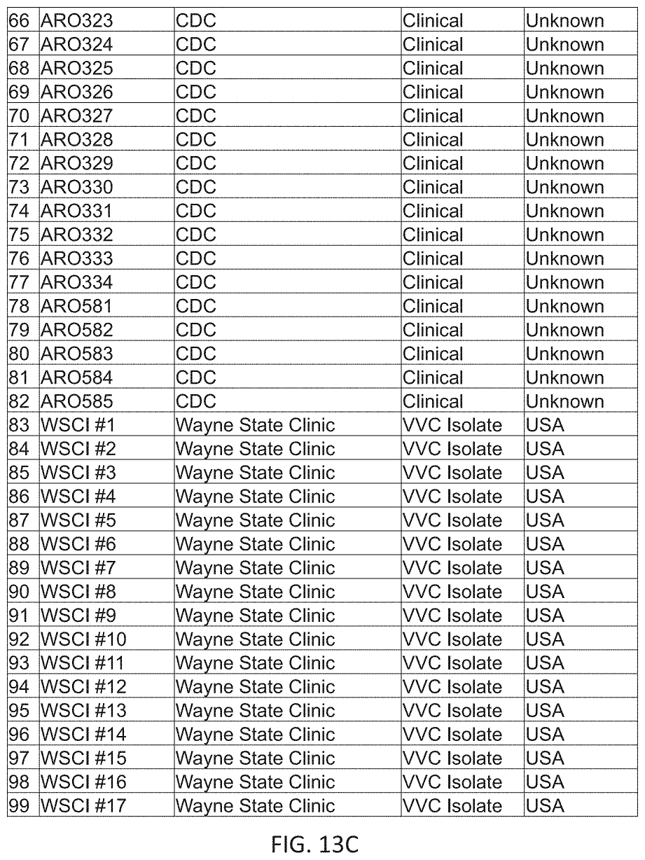

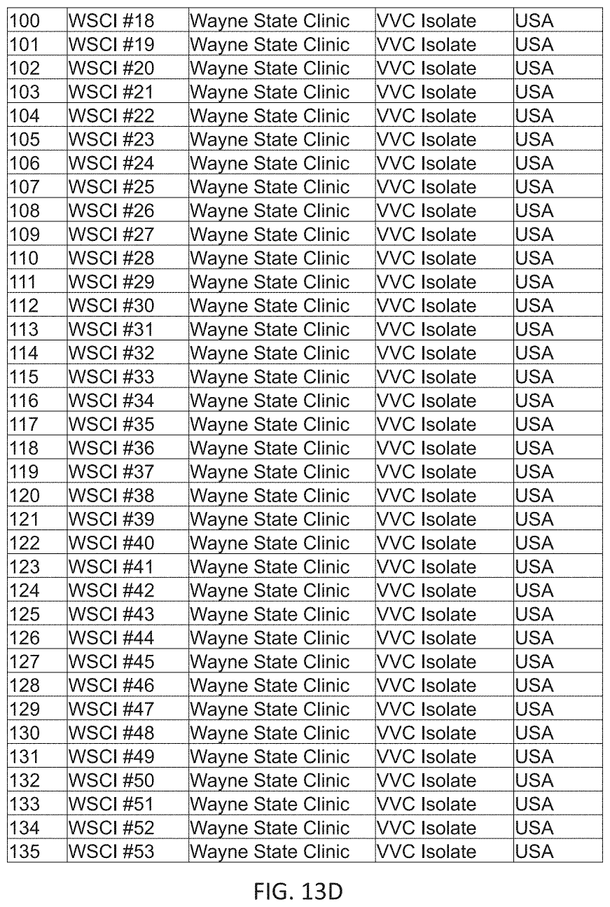

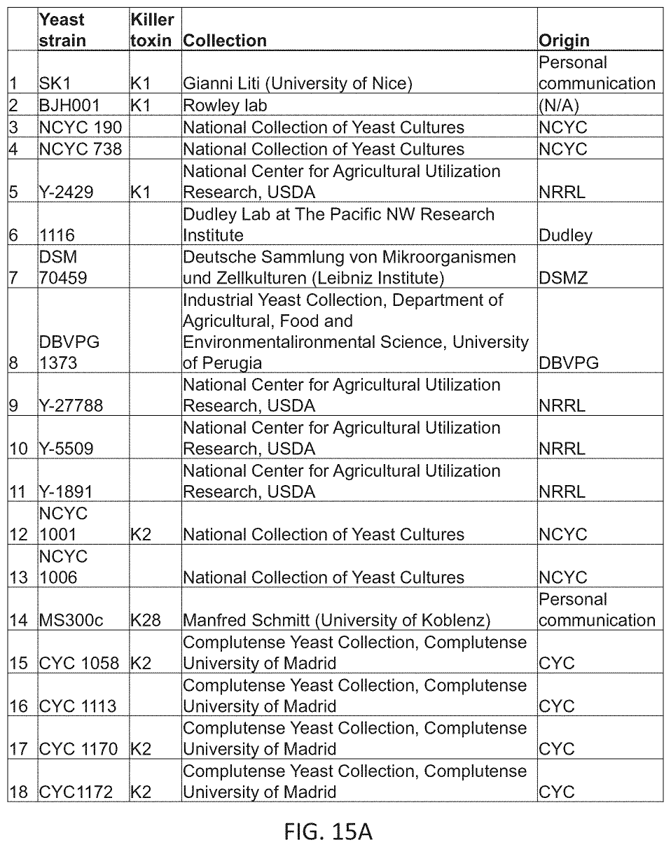

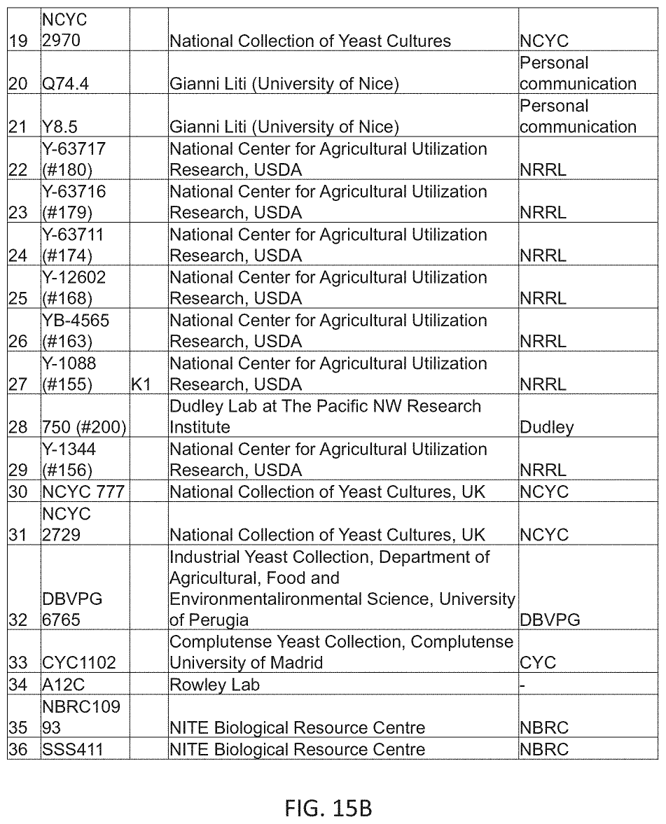

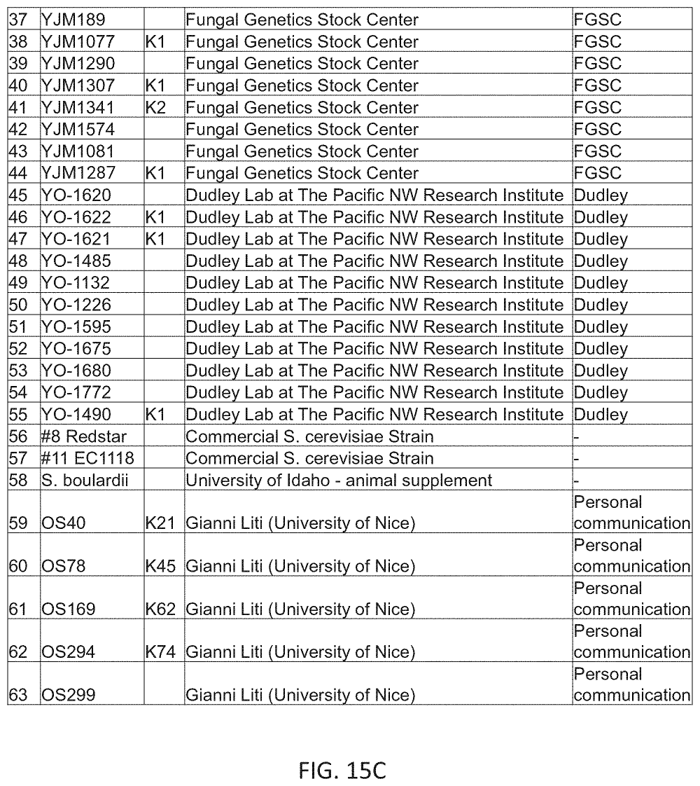

One hundred thirty-five clinical and environmental C. glabrata isolates were obtained from various sources (FIG. 13, Example 1) that encompass all currently known subpopulations of the species as determined by whole genome sequencing. Despite the known strain-specificity of killer toxins expressed by Saccharomyces yeasts, the present study found that 17 tested strains of S. cerevisiae yeasts that express killer toxins (see Example 1) were broadly inhibitory to C. glabrata and were capable of inhibiting the growth of more than 98% of all C. glabrata strains challenged (FIG. 5, Example 1). See FIG. 13 for additional information about the C. glabrata strains shown in FIG. 5, and FIGS. 14-15 for additional information (including killer toxin expressed, if known) about the killer toxin-expressing Saccharomyces yeast strains shown in FIG. 5. Further screens demonstrated that K1- and/or K2-expressing yeasts were broadly antifungal to all 26 clinical strains of C. glabrata provided by the CDC and FDA Antimicrobial Resistance Isolate Bank and also to the 27 environmental and clinical strains provided by the Northern Regional Research Laboratory (Agricultural Research Service Culture Collection) (see Example 1).

A. Killer Toxins

Disclosed herein are killer toxins that may have biological activity against one or more organisms, such as a pathogenic organism. In some embodiments, a killer toxin, such as a K1, K2, and/or K1L toxin or a combination thereof, is expressed by an organism, such as a single-celled and/or multi-celled organism. In certain embodiments, the killer toxin-expressing organism is a fungus, such as a yeast. In a specific, non-limiting example, the organism is a yeast of genus Saccharomyces, such as S. cerevisiae (such as a recombinant S. cerevisiae) or S. paradoxus (such as a recombinant S. paradoxus). In other non-limiting examples, the organism is a recombinant host cell as disclosed herein in Section C. Nucleic Acid Molecules, Vectors, and Host Cells. In additional non-limiting examples, the organism is a host cell as disclosed herein in Section B. Cells.

In some embodiments, the killer toxin has biological activity against a fungus, such as a yeast. In one non-limiting example, the yeast may be a member of genus Candida, such as Candida glabrata. In another non-limiting example, the fungus is a pathogenic organism that can cause VVC, such as Candida glabrata.

A killer toxin of the disclosed embodiments may be a K1, K2, K28, K1us, K74, K21 (also known as K66), K62, K45, K1L, KHR or KHS toxin, or combinations thereof. In some embodiments, the killer toxin is a K1, K2, K28, K1us, K74, K21, K62, K45, or K1L toxin, or combinations thereof. In some embodiments, a killer toxin is a K1 and/or K2 toxin, such as a K1 and/or K2 protein. In some embodiments, a killer toxin is a K1L toxin, such as a K1L protein. In certain embodiments, killer toxins, such as two or more (such as 2, 3, 4, 5, 6, 7, 8, 9, or 10) killer toxins, such as 2, 3, 4, 5, 6, 7, 8, 9, or 10 K1 and/or K2 toxins, comprise amino acid and/or nucleotide sequences having >98% sequence identity (such as, for example, as shown in Table 2).

In any embodiments of the present disclosure, a K1 toxin gene may comprise a sequence having at least 80%, at least 90%, at least 95%, at least 98%, at least 99%, or 100% sequence identity to any one of SEQ ID NOs: 19-30. Additionally, or alternatively, a K1 toxin gene may encode a protein having at least 80%, at least 90%, at least 95%, at least 98%, at least 99%, or 100% sequence identity to any one of SEQ ID NOs: 1-12.

In any embodiments of the present disclosure, a K2 toxin gene may comprise a sequence having at least 80%, at least 90%, at least 95%, at least 98%, at least 99%, or 100% sequence identity to any one of SEQ ID NOs: 31-36. Additionally, or alternatively, a K2 toxin gene may encode a protein having at least 80%, at least 90%, at least 95%, at least 98%, at least 99%, or 100% sequence identity to any one of SEQ ID NOs: 13-18.

In any embodiments of the present disclosure, a K1L toxin gene may comprise a sequence having at least 80%, at least 90%, at least 95%, at least 98%, at least 99%, or 100% sequence identity to SEQ ID NO: 37. Additionally, or alternatively, a K1L toxin gene may encode a protein having at least 80%, at least 90%, at least 95%, at least 98%, at least 99%, or 100% sequence identity to SEQ ID NO: 38. In some embodiments, a killer toxin-expressing organism, such as a yeast, comprises one or more dsRNAs that correspond to one or more viruses and their associated dsRNA satellites. Without being bound to a particular theory, a killer toxin-expressing yeast may comprise two dsRNA molecules, one of which may be a large molecule of about 4,500 base pairs, which may be viral in origin. Such viruses may be of the family Totiviridae, members of which may be widespread within Saccharomyces yeasts. The smaller dsRNAs may be satellite dsRNAs that can encode certain killer toxins that can be expressed by yeasts comprising the dsRNA satellites. The totivirus in the yeast can be responsible for the replication and maintenance of the one or more dsRNA satellites, which can be autonomous genetic elements. The presence of totiviruses and/or dsRNA satellites in a yeast can be confirmed, for example, by determining the molecular weight of one or more dsRNA molecules extracted from a yeast, and/or using reverse transcriptase PCR and/or next-generation sequencing techniques. The genetic sequences of certain killer toxin genes can be determined by extracting one or more dsRNA molecules from yeast strains of interest, and/or by extracting yeast genomic DNA, synthesizing cDNA, and employing short-read sequencing techniques known to persons of ordinary skill in the art. Additional information concerning short-read sequencing techniques can be found in Crabtree A M et al., A Rapid Method for Sequencing Double-Stranded RNAs Purified from Yeasts and the Identification of a Potent K1 Killer Toxin Isolated from Saccharomyces cerevisiae, Viruses, 2019, 11:70, that is incorporated herein by reference in its entirety.

A killer toxin may be used alone, or in combination with one or more additional killer toxins, such as in a combination comprising 2, 3, 4, 5, or more killer toxins. Killer toxins are expressed by organisms and a person of ordinary skill of the art understands that as used herein killer toxins are understood to be at least partially separated from such organisms or cellular fragments thereof. Killer toxins have a purity of at least 50%, at least 60%, at least 70%, at least 80%, at least 90%, at least 95%, at least 96%, at least 97%, at least 98%, at least 99%, or substantially 100%. Methods of isolating and/or purifying such molecules are known in the art, and this disclosure is not limited to one or any specific method. Exemplary non-limiting methods include those that employ ethanol precipitation and/or ammonium sulfate (see Example 3). Additional exemplary non-limiting methods include size-exclusion chromatography, affinity chromatography, cellulose affinity chromatography, ultracentrifugation, ultrafiltration, and centrifugal partition chromatography.

In some embodiments, one or more killer toxins are provided in combination with (such as in a composition with) one or more additional components, such as, for example, one or more killer toxin-expressing organisms (such as a killer toxin-expressing yeast, such as a killer toxin-expressing S. cerevisiae or a killer toxin-expressing S. paradoxus), one or more pharmaceutically acceptable excipients (such as one or more carriers, buffers, adjuvants, emulsifiers, salts, and/or other therapeutics), or combinations thereof. In some embodiments, one or more killer toxins are provided as part of a kit, and/or in combination with one or more apparatuses for administering the one or more killer toxins to a subject in need thereof, one or more media for culturing one or more organisms (such as one or more isolated organisms), one or more components useful for testing the efficacy of the one or more toxins against one or more organisms, or combinations thereof.

B. Organisms

Certain embodiments of the present disclosure concern compositions comprising one or more killer toxins and/or one or more organisms that express the one or more killer toxins, and methods of using such compositions. Thus, disclosed herein are organisms comprising one or more cells (such as a single-celled organism or a multicellular organism) that express one or more killer toxins that may have biological activity against one or more other organisms, such as a pathogenic organism.

In some embodiments, the organism that expresses one or more killer toxins is a fungus. In one non-limiting example, the organism is a yeast, such as, for example, a yeast of genus Saccharomyces or genus Pichia, such as a recombinant yeast of genus Saccharomyces or genus Pichia. In specific non-limiting examples, the yeast is Pichia pastoris, Saccharomyces cerevisiae, or Saccharomyces paradoxus. In another non-limiting example, the organism is a non-yeast fungus, such as a filamentous fungus. In certain embodiments, the non-yeast fungus is a member of genus Aspergillus, Trichoderma, Penicillium, Ashbya, Phanerochaete, Chrysosporium, Fusarium, or Rhizopus. In specific non-limiting examples, the fungus is Trichoderma reesei, Aspergillus niger, Aspergillus oryzae, Ashbya gossypii, Fusarium oxysporum, Phanerochaee chrysosporium, or Chrysosporium lucknowense. In another non-limiting example, the organism is a prokaryote, such as, for example, a bacterium of genus Vibrio, Salmonella, Escherichia, Lactococcus, Listeria, Lactobacillus, Shigella, Bordetella, Bacillus, Mycobacterium, Yersinia, or Pseudomonas. In specific non-limiting examples, the bacterium is Bacillus subtilis, Salmonella enterica, Escherichia coli, Lactococcus lactis, Listeria monocytogenes, Mycobacterium smegmatis, Pseudomonas aeruginosa, Shigella flexneri, Yersinia enterocolitica, Mycobacterium bovis, or Vibrio anguillarum. In another non-limiting example, the organism is an animal, such as an animal cell, such as a mammalian cell or an avian cell. In specific non-limiting examples, the cell is a Chinese hamster ovary (CHO) cell, a murine myeloma (NSO) cell, a human embryonic kidney (HEK) cell, or a baby hamster kidney (BHK) cell. In another specific non-limiting example, one or more toxins are collected from an avian egg, such as a chicken (Gallus gallus) egg, such as an egg of a transgenic avian, such as a transgenic chicken. In another specific non-limiting example, one or more killer toxins are collected from a transgenic sheep, goat, or cow, such as from milk collected from the transgenic animal.

In some embodiments, the one or more toxins expressed by the organism include K1, K1us, K62, K45, K74, Y8.5, K28, K21, K2, K1L, KHR, or KHS toxins, or combinations thereof. In specific non-limiting examples, the organism expresses one or more K1 and/or K2 toxins. In some embodiments of the present disclosure, an organism expresses one or more K1 and/or K2 genes as described herein.

In specific non-limiting examples, an organism that expresses the one or more killer toxins is a yeast of genus Saccharomyces, such as S. cerevisiae or S. paradoxus. In another non-limiting example, S. cerevisiae cells express killer toxins with biological activity against a member of genus Candida, such as C. glabrata. In some embodiments, S. cerevisiae cells and/or S. paradoxus cells express killer toxins with biological activity against multiple C. glabrata strains, for example, against C. glabrata strains capable of infecting and/or causing disease (such as VVC) in an animal, such as a human subject. In some examples, the disclosed toxins expressed by the organism inhibit the growth of one or more C. glabrata strains that are resistant to clinically relevant antifungal drugs, such as, for example, azole and/or echinocandin drugs.

Organism strains and/or species that express one or more killer toxins as disclosed herein may be used individually or in combination, such as two, three, four, five, or more strains and/or species. For example, a composition comprising a single species that produces one or more disclosed killer toxins may comprise one strain of that species, or it may comprise multiple, such as 2, 3, 4, 5, or more strains of the species. Additionally, or alternatively, embodiments of the disclosed composition may comprise multiple species that each produce one or more toxins as disclosed herein, and may further comprise one or more, such as 2, 3, 4, 5 or more, strains of each species. Cells disclosed herein that express one or more killer toxins may be of one or more strains of one or more species, such as, for example, one or more S. cerevisiae strains, one or more S. paradoxus strains, and one or more P. pastoris strains. Exemplary, non-limiting S. cerevisiae strains disclosed herein that express antifungal toxins with biological activity against one or more C. glabrata strains are provided in Table 2. In certain embodiments, the one or more cells of one or more species and/or strains are yeast cells, such as a Saccharomyces yeast, such as S. cerevisiae, such as S. cerevisiae strain YO-1622, YO-1482, YJM1287, YJM1307, Y-2429, YO-1490, SK1, YJM1077, CYC1058, CYC1170, YJM1341, NCYC1001, or CYC1172, or combinations thereof. In some embodiments, the one or more cells of one more species and/or strains are S. paradoxus cells. Certain embodiments of the killer toxin-expressing cells disclosed herein comprise isolated cells, such as an isolated yeast of genus Saccharomyces, such as an isolated S. cerevisiae (such as an isolated S. cerevisiae strain YO-1622), or an isolated S. paradoxus.

TABLE 2
Exemplary S. cerevisiae strains that express killer
toxins with biological activity against one or more strains
of C. glabrata. (See also FIGs. 14-15)

| | | | % amino acid |
|---|---|---|---|
| | | | identity to |
| Toxin | | | canonical |
| type | Strain | Source | killer toxins |
| K1 | BJH001 | Rowley Lab | 99.38 |
| K1 | YO-1622 | Aimée Dudley, Ph.D. | 98.31 |
| | | Pacific Northwest Research | |
| | | Institute, Seattle, WA | |
| | | (Dudley lab PNRI) | |
| K1 | YO-1482 | Dudley lab PNRI | 98.31 |
| K1 | YJM1287 | Fungal Genetics Stock Center | 99.32 |
| | | Department of Plant Pathology | |
| | | Kansas State University | |
| | | Manhattan, KS | |
| K1 | YJM1307 | Fungal Genetics Stock Center | 99.66 |
| K1 | Y-2429 | Agricultural Research Service | 99.32 |
| | | Culture Collection (NRRL) | |
| K1 | YO-1490 | Dudley lab PNRI | Not determined |
| | | | (truncated |
| | | | sequence) |
| K1 | SK1 | Fungal Genetics Stock Center | Not determined |
| | | | (truncated |
| | | | sequence) |
| K1 | YJM1077 | Fungal Genetics Stock Center | Not determined |
| | | | (truncated |
| | | | sequence) |
| K2 | CYC1058 | Complutense Yeast Collection, | 98.31 |
| | | University of Madrid | |
| K2 | CYC 1170 | Complutense Yeast Collection, | 98.87 |
| | | University of Madrid | |
| K2 | YJM1341 | Fungal Genetics Stock Center | Not determined |
| | | | (truncated |
| | | | sequence) |
| K2 | NCYC1001 | National Collection of Yeast | Not determined |
| | | Cultures | (truncated |
| | | | sequence) |
| K2 | CYC 1172 | Complutense Yeast Collection, | Not determined |
| | | University of Madrid | (truncated |
| | | | sequence) |

Killer toxin-expressing organisms disclosed herein, such as yeasts, may endogenously express one or more killer toxins as disclosed herein, or may be recombinant organisms (such as recombinant yeasts) that express one or more killer toxins. Such recombinant organisms may also express one or more killer toxins endogenously, in addition to expressing one or more killer toxins as a result of cellular transformation, for example with a nucleic acid molecule (such as a plasmid) encoding one or more killer toxins. Certain organisms disclosed herein may comprise one or more copies of one or more full or partial genes encoding the same killer toxin or different killer toxins in various combinations.

Thus, also disclosed herein are embodiments of a recombinant (i.e., transformed) organism, such as a recombinant yeast, such as a recombinant S. cerevisiae or a recombinant S. paradoxus, that comprise one or more exogenous nucleic acid molecules (such as a plasmid) encoding one or more killer toxins, such as one or more K1, K2, and/or K1L toxins. In some embodiments, a recombinant (i.e., transformed) organism, such as a recombinant S. cerevisiae or a recombinant S. paradoxus, comprises one or more exogenous nucleic acid molecules encoding at least one (such as at least 1, at least 2, at least 3, at least 4, at least 5, at least 6, at least 7, or at least 8) K1 toxin, at least one (such as at least 1, at least 2, at least 3, at least 4, at least 5, at least 6, at least 7, or at least 8) K2 toxin, and/or at least one (such as at least 1, at least 2, at least 3, at least 4, at least 5, at least 6, at least 7, or at least 8) K1L toxin.

In some embodiments, a recombinant organism disclosed herein comprises at least two (such as at least 2, at least 3, at least 4, at least 5, at least 10, at least 15 or at least 20) copies, such as at least two copies having the same sequence, of one or more killer toxin genes, such as one or more K1, K2, and/or K1L genes.

In some embodiments, a disclosed organism, such as an S. cerevisiae or an S. paradoxus, prior to transformation with one or more nucleic acid molecules (such as a plasmid) comprising one or more killer toxins (such as one or more K1, K2, and/or K1L toxins), does not express a killer toxin, or expresses one or more killer toxins having a nucleic acid and/or amino acid sequence that differs from the exogenous sequence or sequences.

In specific non-limiting embodiments, a disclosed organism, prior to transformation with one or more nucleic acid molecules comprising one or more exogenous toxins, such as one or more exogenous K1 and/or K2 toxins, expresses one or more additional antifungal toxins, such as, for example, K21, K28, K45, K1us, K74, K62, K1L, KHR, KHS, a different or the same K1, a different or the same K2, or combinations thereof.

In one specific non-limiting example, a recombinant organism, such as a recombinant S. cerevisiae, that comprises at least one endogenous K1, K2, and/or K1L toxin gene further comprises one or more exogenous K1, K2, and/or K1L toxin genes.

C. Nucleic Acid and Molecules, Vectors, and Host Cells

Multiple types and versions of vectors, nucleic acid molecules, and cells comprising one or more killer toxins, such as killer toxins as disclosed herein, for example, one or more K1, K2, and/or K1L toxins, also are within the scope of the present disclosure. Thus, disclosed herein are nucleic acid molecules encoding killer toxins, such as K1, K2, and/or K1L toxins. K1,K2, and K1L toxin protein and nucleic acid sequences are publicly available and specific examples are provided herein. In addition, K1, K2, and K1L sequences can be identified using molecular biology methods. Exemplary K1 nucleotide sequences are provided in SEQ ID NOs: 19-30, and exemplary K1 amino acid sequences are provided in SEQ ID NOs: 1-12. Exemplary K2 nucleotide sequences are provided in SEQ ID NOs: 31-36, and exemplary K2 amino acid sequences are provided in SEQ ID NOs: 13-18. An exemplary K1L nucleotide sequence is provided in SEQ ID NO: 37, and an exemplary K1L amino acid sequence is provided in SEQ ID NO: 38. A person of ordinary skill in the art understands that variants of the K1, K2, and K1L nucleic acid sequences provided herein can be introduced into an organism as disclosed herein, such as a Saccharomyces cerevisiae yeast.

Exogenous killer toxin genes can be part of one or more exogenous nucleic acid molecules (such as 1, 2, 3, 4, 5, 6, 7, 8, 9, or 10 exogenous nucleic acid molecules). In some examples, exogenous nucleic acid molecules can be part of a vector, such as a plasmid or viral vector. In some examples, expression of the exogenous nucleic acid molecules is driven by one or more promoters, such as a constitutive or inducible promoter, or a bi-directional promoter. In some examples, the promoter used to drive expression of one or more exogenous nucleic acid molecules is a native promoter (e.g., native to a K1 K2, and/or K1L gene expressed). In other examples, the promoter used to drive expression of the one or more nucleic acid molecules is a non-native promoter (e.g., exogenous to a K1, K2, and/or K1L gene expressed). In one specific non-limiting example, dsRNAs encoding one or more killer toxins can be cloned from the K1-expressing YJM1307 and YO1622 S. cerevisiae strains and from the K2-expressing CYC1172 and YJM1341 S. cerevisiae strains, incorporated into one or more yeast plasmid vectors, and ectopically expressed individually or in combinations in a strain that does not otherwise express killer toxins, such as BY4741, or in a strain that expresses the same or different killer toxins. In another specific non-limiting example, the BY4741 strain, which previously did not express K1, K2, or K1L toxins, can be made to express one or more functional K1, K2, and/or K1L toxins (such as one or more K1 toxins having different nucleic acid and/or amino acid sequences, one or more K2 toxins having different nucleic acid and/or amino acid sequences, one or more K1L toxins having different nucleic acid and/or amino acid sequences, multiple copies of a K1 toxin having the same nucleic acid and/or amino acid sequence, multiple copies of a K2 toxin having the same nucleic acid and/or amino acid sequence, or combinations thereof, and/or multiple copies of a K1L toxin having the same nucleic acid and/or amino acid sequence, or combinations thereof) as desired.

Methods of producing the vectors, nucleic acid molecules, and cells disclosed herein are known to those of ordinary skill in the art, and the disclosure is not limited to using one or more specific vector, nucleic acid molecule, or host cell production methods, or to specific vectors, nucleic acid molecules, or cell types. For example, vectors and host cells that comprise one or more K1, K2, and/or K1L toxins comprise one or more nucleic acid molecules encoding the one or more K1, K2, and/or K1L toxins, such as any one or more of the sequences of SEQ ID NOs. 1-38, and recombinant host cells are typically generated to express the one or more toxins. Naked nucleic acid molecules, such as, for example, a plasmid, are typically produced to express one or more killer toxins, such as one or more K1, K2, and/or K1L toxins, for example of any one or more of SEQ ID NOs. 1-38, following cellular transformation with one or more such nucleic acid molecules (such as transformation of one or more S. cerevisiae strains to express one or more K1, K2, and/or K1L toxins). Thus, one or more compositions comprising at least one vector, nucleic acid molecule, and/or host cell described herein, or combinations thereof, can be administered to a subject to, for example, treat VVC, such as to ameliorate or eliminate one or more symptoms associated with VVC, and/or to inhibit the growth of and/or cause the death of cells of the organism (such as C. glabrata) causing the VVC.

Also disclosed herein are vectors, such as plasmid vectors, viral vectors, and/or host cells, comprising one or more toxins as disclosed herein, such as one or more K1, K2, and/or K1L toxins. In some embodiments, the viral vector can be a herpesvirus, adenovirus, adeno-associated virus (AAV), alphavirus, retrovirus, lentivirus, flaviviruses, measles viruses, rhabdoviruses, Newcastle disease virus (NDV), poxviruses, picornaviruses, a chimeric virus, or any combination thereof. In some embodiments, one or more killer toxins are expressed using a baculovirus expression system. A person of ordinary skill in the art will understand how to use vectors, such as for expression of a killer toxin in a recombinant organism and/or host cell. Additional information concerning yeast vectors that can be used in disclosed embodiments can be found in Alberti et al. (2007), A suite of Gateway cloning vectors for high-throughput genetic analysis in Saccharomyces cerevisiae, Yeast, 24(10):913, that is incorporated herein by reference in its entirety.

In some embodiments, one or more nucleic acid molecules encoding one or more K1, K2, and/or K1L toxins, such as one or more toxins of SEQ ID NOs. 1-38, may be incorporated into a host cell. In one non-limiting example, the host cell is a recombinant yeast, such as, for example, a yeast of genus Saccharomyces or genus Pichia. In specific non-limiting examples, the recombinant yeast is Pichia pastoris, Saccharomyces cerevisiae, or Saccharomyces paradoxus. In another non-limiting example, the host cell is a non-yeast fungus, such as a filamentous fungus, such as, for example, an Aspergillus, Trichoderma, Penicillium, Ashbya, Phanerochaete, Chrysosporium, Fusarium, or Rhizopus species. In specific non-limiting examples, the filamentous fungus is Trichoderma reesei, Aspergillus niger, Aspergillus oryzae, Ashbya gossypii, Fusarium oxysporum, Phanerochaee chrysosporium, or Chrysosporium lucknowense. In another non-limiting example, the host cell is a recombinant prokaryote, such as, for example, a bacterium of genus Vibrio, Salmonella, Escherichia, Lactococcus, Listeria, Lactobacillus, Shigella, Bordetella, Bacillus, Mycobacterium, Yersinia, or Pseudomonas. In specific non-limiting examples, the recombinant bacterium is Bacillus subtilis, Salmonella enterica, Escherichia coli, Lactococcus lactis, Listeria monocytogenes, Mycobacterium smegmatis, Pseudomonas aeruginosa, Shigella flexneri, Yersinia enterocolitica, Mycobacterium bovis, or Vibrio anguillarum. One or more of the nucleic acid molecules can be incorporated into a host cell by one of several techniques by which a nucleic acid molecule might be introduced into a cell. Techniques, such as, for example, transformation with a plasmid encoding one or more toxins, such as one or more toxins of SEQ ID NOs. 1-38, are known to a person of ordinary skill in the art. In one specific non-limiting example, a plasmid encoding one or more, such as, for example, 1, 2, 3, 4, 5, 6, 7, 8, 9, or 10 toxins, such as any one or more toxins of SEQ ID NOs. 1-38, is incorporated into a Saccharomyces cerevisiae host cell, and a composition comprising the transformed host cell is administered to a subject.

### III. Compositions and Kits

Provided herein are compositions that comprise one or more killer toxins, one or more organisms expressing one or more killer toxins, one or more nucleic acid molecules, vectors, and/or host cells comprising one or more killer toxins, and combinations thereof. In certain embodiments, compositions comprise one or more organisms, such as a yeast, such as a Saccharomyces yeast, such as S. cerevisiae and/or S. paradoxus, that express one or more killer toxins, such as K1, K2, and/or K1L toxins, such as compositions for administration of the one or more organisms to a subject in need thereof. Also provided are compositions that comprise one or more killer toxins, such as one or more K1, K2, and/or K1L toxins, such as compositions for administration of the one or more killer toxins to a subject in need thereof. Certain embodiments of disclosed compositions comprise one or more killer toxins and one or more organisms expressing the same or different one or more killer toxins. Certain embodiments of compositions comprise one or more isolated (such as purified) killer toxins, and/or one or more isolated (such as purified) organisms expressing one or more killer toxins, and/or one or more isolated (such as purified) nucleic acid molecules, vectors, and/or host cells comprising one or more killer toxins.

Certain embodiments of the disclosed compositions are stable at human body temperatures. In some embodiments, compositions disclosed herein can be stored prior to use (such as prior to administration to a subject) at temperatures at, about, or below freezing (such as at, about, or below 0° C., such as −4° C., such as −20° C., such as −80° C.), at temperatures at or about room temperature (such as at or about 25° C.), at temperatures above room temperature (such as at, about, or above 37° C.), or at any temperature ranging between about −80° C. and about 40° C. Certain embodiments of disclosed compositions can be stored in liquid nitrogen.

Some embodiments of disclosed compositions may be a formulation, such as an ointment, cream, suppository, solid (such as a capsule, ovule, tablet, pill, and/or suppository), solution, suspension, foam, film, gel, liposomal composition, or combinations thereof. Certain embodiments of the disclosed compositions may also comprise an applicator, such as a tampon, vaginal ring, pillow, puff, sponge, osmotic pump system, an applicator useful for administering (such as topically and/or vulvovaginally) a cream and/or ointment, an applicator useful for administering (such as intravaginally) a capsule, ovule, tablet, pill, and/or suppository, or combinations thereof. Compositions may comprise conventional components of such forms of administration, which are known to those of ordinary skill in the art. In certain embodiments, a composition comprises at least one component that is bioadhesive to the vulvovaginal surface. In some embodiments, a composition is adapted for application of a unit dose amount to a vulvovaginal surface. In certain embodiments, a composition comprises one or more additional components selected from the group consisting of a pharmaceutically acceptable excipient, an adjuvant, a carrier, salt, another therapeutic, and combinations thereof.

Exemplary adjuvants include, but are not limited to, aluminum hydroxide, alum, aluminum phosphate, killed bacteria, squalene, detergents, cytokines, paraffin oil, and combination adjuvants, such as Freund's complete adjuvant or Freund's incomplete adjuvant. Excipients can include, but are not limited to, antiadherents, binders, coatings, enteric coatings, disintegrants, flavorings, sweeteners, colorants, lubricants, moisturizing agents, glidants, sorbents, preservatives, adjuvants, carriers (such as suspension aids and/or solubilizing aids), or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

Compositions comprising one or more organisms (such as a yeast, such as a Saccharomyces yeast, such as S. cerevisiae) expressing one or more killer toxins, and/or one or more killer toxins (such as K1 and/or K2 toxins), can include one or more component selected from water (such as purified water), maltodextrin, lactose, fructose and fructose derivatives, short chain fructo-oligosaccharides, fatty acids, lactic acid, stearic acid, PPG-18, PEG-18, PEG-20, PEG-32, PEG-40, silica, methylparaben, aloe vera (such as Aloe leaf extract, such as Aloe barbadensis leaf extract), anti-inflammatory compounds, vitamins, minerals, amino acids, lubricants, hydrolyzed cellulose, hydroxypropyl cellulose, hypromellose, cellulose, microcrystalline cellulose, gelatin, magnesium stearate, titanium dioxide, ascorbic acid, benzoic acid, cetyl alcohol, stearyl alcohol, isopropyl myristate, cetyl ethylhexanoate, polysorbate (such as polysorbate 60 or polysorbate 20), povidone, trolamine, potassium hydroxide, propylene glycol, caprylyl glycol, ceteareth-20, glyceryl oleate, acrylates, cyclopentasiloxane, EDTA, disodium EDTA, cyclopentasiloxane, trideceth-6, ethanol, dicalcium phosphate, sodium hydroxide, sodium citrate, citric acid, potassium citrate, a hydrocarbon propellant (such as propane and/or butane), such as a hydrocarbon propellant for a composition administered in the form of a foam or spray, sorbic acid, tocopheryl acetate, phenoxyethanol, Hamamelis (such as Hamamelis virginiana) plant extract, vegetable oil, coconut oil, fractionated coconut oil, palm oil, fractionated palm oil, castor oil, hydrogenated castor oil, glyceryl dilaurate, dimethicone, colloidal oatmeal, sodium polyacrylate, one or more Federal Food, Drug, and Cosmetic Act colorants approved for use in animals (such as humans), or combinations thereof.

Compositions comprising one or more killer toxins (such as K1, K2, and/or K1L toxins) and/or one or more organisms (such as yeasts, such as Saccharomyces yeasts, such as one or more S. saccharomyces yeasts) expressing one or more killer toxins can comprise one or more other therapeutics, such as, but not limited to, other therapeutics for treating a yeast infection, such as VVC. Exemplary other therapeutics can include clotrimazole, econazole, miconazole, terbinafine, fluconazole, ketoconazole, amphotericin (such as amphotericin B, amphotericin B cholesteryl sulfate, amphotericin B deoxycholate, amphotericin B liposomal, and/or amphotericin B phospholipid complex), nystatin, terbinafine, voriconazole, posaconazole, griseofulvin, itraconazole, isavuconazole, itraconazole, flucytosine, metronidazole, clindamycin, tinidazole, and combinations thereof. Additionally, or alternatively, other therapeutics known in the art, such as, but not limited to, one or more antibiotics, anti-inflammatories, lubricants, antivirals, anticoagulants, steroids, anticancer agents, or combinations thereof, may be administered in combination with one or more of the killer toxins and/or organisms disclosed herein.

Also contemplated are kits that comprise one or more killer toxins, one or more organisms (such as an organism expressing one or more killer toxins and/or an organism that can be transformed to express one or more killer toxins), one or more nucleic acid molecules, vectors, and/or host cells comprising one or more killer toxins, and combinations thereof. A kit of the present disclosure can comprise any one or more organisms as disclosed in herein; any one or more killer toxins as disclosed herein; and/or any one or more nucleic acid molecules, vectors, and/or host cells expressing one or more killer toxins as disclosed in the present disclosure. Certain embodiments of kits disclosed herein comprise one or more isolated (such as purified) killer toxins, and/or one or more isolated (such as purified) organisms expressing (or that can be transformed to express) one or more killer toxins, and/or one or more isolated (such as purified) nucleic acid molecules, vectors, and/or host cells comprising one or more killer toxins. In a specific non-limiting example, a kit comprises one or more yeasts, such as one or more Saccharomyces yeasts, such as one or more S. cerevisiae strains, that express one or more killer toxins. In another specific, non-limiting example, a kit comprises one or more organisms and one or more nucleic acid molecules and/or vectors comprising one or more killer toxins that can be used to transform the one or more organisms to express the one or more killer toxins.

In some examples, either prior to use or prior to and during use, one or more organisms that express (or can be transformed to express) one or more toxins are freeze dried, lyophilized, and/or stored in a solution comprising glycerol.

In some embodiments, a kit may comprise one or more additional components, such as, for example, one or more components that permit culturing of one or more organisms that express (or can be transformed to express) one or more killer toxins (such as one or more media and/or one or more containers for culturing the one or more organisms), one or more components useful for testing the efficacy of one or more killer toxins (or an organism expressing one or more killer toxins) against one or more organisms, and/or a description of the component or components that permit culturing of the one or more organisms and/or testing of the one or more killer toxins or one or more organisms that express one or more killer toxins. In other embodiments, a kit may comprise one or more components for administering one or more organisms as disclosed herein (such as an organism expressing one or more killer toxins, or an organism that can be transformed to express one or more killer toxins) and/or one or more killer toxins as disclosed herein, and/or instructions for administering the one or more organisms and/or one or more killer toxins. In some embodiments, a kit comprises an applicator for administering one or more toxins (such as K1, K2, and/or K1L toxins), and/or one or more organisms that express one or more toxins (such as one or more Saccharomyces strains, such as one or more S. cerevisiae strains or one or more S. paradoxus strains) to a subject in need thereof.

In some embodiments, a disclosed kit includes one or more media for culturing, storing, and/or growing one or more organisms, such as one or more yeasts, such as one or more Saccharomyces yeasts, such as one or more S. cerevisiae strains or one or more S. paradoxus strains. Exemplary media that can be included in the disclosed compositions and kits include solid media (such as those containing agar, for example complete medium or minimal medium) and/or liquid media (such as a fermentation broth, such as complete medium or minimal medium). Media can also be provided in kits and/or compositions in dry powder form for rehydration prior to use. Media that can be used to culture organisms included in compositions and kits disclosed herein are known in the art. In some embodiments, a kit or composition includes yeast extract/peptone/dextrose (YPD) medium, vaginal-simulative medium, complete supplement mixture medium, yeast peptone medium, yeast nitrogen base medium. In some embodiments, a kit or composition includes Luria-Bertani agar medium, Luria-Bertani broth, enriched media, tryptic soy broth, MacConkey agar, and/or S.O.C. medium. Kits or compositions can also comprise one or more media additives, such as, for example, agar, peptone, ammonium sulfate, yeast extract, salts, trace elements, vitamins, sodium citrate (for example for buffering), potassium hydroxide, calcium hydroxide, lactic acid, acetic acid, urea, glycerol one or more nitrogen sources, one or more amino acids, one or more sugars (such as glucose, galactose, raffinose, and/or dextrose), one or more antibiotics (such as for selection of transformed, killer toxin-expressing cells), or combinations thereof. Kits can also comprise one or more containers, such as single- or multi-well plates, for culturing one or more organisms, and/or for testing the efficacy of one or more killer toxins and/or one or more organisms expressing one or more killer toxins against one or more other organisms. Containers may also include airtight gas chambers for incubation of cultures under hypoxic conditions (such as, but not limited to, conditions comprising 2% oxygen, 5% CO₂, and 93% nitrogen). Vaginal-stimulatory media and conditions are known to those of ordinary skill in the art. However, additional information can be found in Owen & Katz (1999), A vaginal fluid simulant, Contraception, 59:91-95, that is incorporated by reference herein in its entirety.

### IV. Methods of Administration

In some embodiments, compositions disclosed herein may be administered to a subject to treat or prevent a yeast infection, such as a C. glabrata infection. Administration may be any suitable route known to persons of ordinary skill in the art, such as parenteral, intramuscular, and/or subcutaneous injection, oral administration, topical administration, and/or vulvovaginal administration, such as intravaginal administration and/or vulvar administration, such as, for example, for treating or preventing an infection, such as a yeast infection (such as VVC), such as a yeast infection caused by a yeast of genus Candida, such as a yeast infection caused by Candida albicans. Compositions administered herein can further comprise one or more pharmaceutically acceptable excipients, such as a buffer, carrier, adjuvant, pharmaceutically acceptable salt, carrier, or combinations thereof, suitable for administration to a vaginal and/or vulvar surface. In some embodiments the method of administration comprises administering a composition comprising a killer toxin consisting of a K1 protein, a K2 protein, a K1L protein, or a combination thereof, and one or more formulation components, such as one or more pharmaceutically acceptable excipients. Administration may comprise providing to a subject one or more compositions disclosed herein in the form of an ointment, cream, suppository, solid (such as a capsule, ovule, tablet, pill, or suppository), solution, suspension, foam, film, gel, liposomal composition, or combinations thereof. Compositions administered herein may also be administered using a tampon, vaginal ring, pillow, puff, sponge, osmotic pump system, an applicator useful for administering (such as topically and/or vulvovaginally) a cream and/or ointment, an applicator useful for administering (such as intravaginally) a capsule, ovule, tablet, pill, and/or suppository, or combinations thereof. In some embodiments, the compositions may be administered using one or more applicators. In other embodiments, no applicator is required for administration.

Compositions comprising one or more organisms (such as one or more yeasts) that express one or more killer toxins (such as one or more K1, K2, and/or K1L toxins), and/or one or more killer toxins (such as one or more K1, K2, and/or K1L toxins, such as one or more isolated, K2, and/or K1L toxins), and/or one or more nucleic acid molecules, vectors, and/or host cells comprising one or more killer toxins, are typically used in an amount effective to achieve an intended result, for example, in an amount effective to treat, prevent, or ameliorate VVC caused by C. glabrata in a subject. One or more organisms that express one or more killer toxins, and/or one or more killer toxins, and/or one or more nucleic acid molecules, vectors, and/or host cells comprising one or more killer toxins, or compositions comprising one or more such organisms, killer toxins, nucleic acid molecules, vectors, and/or host cells, can be administered therapeutically to achieve therapeutic benefit or prophylactically to achieve a prophylactic benefit. Exemplary benefits may include, but are not limited to, eradication and/or amelioration of the underlying condition, (such as a VVC, such as a VVC caused by C. glabrata) being treated, and/or eradication or amelioration of one or more symptoms associated with the condition (such as one or more symptoms associated with VVC, such as, but not limited to, itching and irritation in the vagina and/or vulva, a burning sensation, redness and/or swelling of the vulva, vaginal pain and/or soreness, vaginal rash, and/or vaginal discharge). Benefits may include, but are not limited to, that the patient reports an improvement in feeling or condition, notwithstanding that the patient may still be afflicted with the underlying condition, such as a VVC. For example, administration of an organism expressing one or more killer toxins, and/or one or more killer toxins to a subject suffering from C. glabrata-caused VVC provides therapeutic benefit when the underlying VVC is eradicated or ameliorated, and/or when the subject reports a decrease in the severity or duration of the symptoms associated with the VVC. Therapeutic benefit can also comprise halting or slowing the progression of the condition, such as the VVC, regardless of whether improvement is realized.

As known by those of ordinary skill in the art, preferred time, dose, and/or route of administration, such as administration to a subject, of protein-based therapeutics, such as the disclosed killer toxins, and/or organisms expressing one or more protein-based therapeutics, such as organisms expressing one or more killer toxins as disclosed (such as an S. cerevisiae strain expressing one or more killer toxins), may depend on various factors, including subject age, body weight, diet, and/or general health, and/or the condition being treated, the severity and/or stage of the condition being treated, the form of the composition being administered, and/or the type and/or concentration of one or more components of the composition being administered.

Dosage may also be tailored to subjects suffering from more than one condition, such as those subjects who have one or more additional vulvovaginal infections. In certain specific examples, dosage and frequency of administration of one or more killer toxins (such as one or more K1 and/or K2 toxins) and/or one or more Saccharomyces yeasts expressing one or more killer toxins, or compositions thereof, will also depend on whether the yeasts and/or toxins are formulated for treatment of acute episodes of VVC or for the prophylactic treatment of VVC, for example in a subject susceptible to VVC. One of ordinary skill in the art will be able to determine the optimal dose and timing of administration for a particular subject.

For prophylactic administration of one or more killer toxins (such as one or more K1, K2, and/or K1L toxins) and/or one or more organisms (such as one or more Saccharomyces yeasts) expressing one or more killer toxins, or compositions thereof, can be administered to a patient or subject at risk of developing a condition, for example, VVC. Alternatively, prophylactic administration can be used to avoid or ameliorate the onset of symptoms in a patient diagnosed with a condition, such as VVC. For example, one or more killer toxins (such as one or more, K2, and/or K1L toxins) and/or one or more organisms (such as one or more Saccharomyces yeasts) expressing one or more killer toxins, or composition thereof, can be administered to a subject prior to an event or treatment that may increase susceptibility of the subject to a condition (such as a VVC), such as, for example, administration of an anti-bacterial therapeutic.

Effective dosages can be estimated initially from in vitro assays. Initial dosages can also be estimated from in vivo data, such as animal models. Animal models useful for testing the efficacy of organism- (such as yeasts, such as Saccharomyces yeasts, such as S. cerevisiae) and/or protein-based therapeutics to treat or prevent various conditions and diseases are known in the art. Such models include, but are not limited to, mice, rats, macaques, zebrafish, Galleria mellonella, Drosophila melanogaster, Caenorhabditis elegans, Panagrellus redivivus, and Manduca sexta. One of ordinary skill in the art can adapt such information to determine dosages and timing of dosages of, for example, compositions disclosed herein, suitable for administration in a human subject. Additional information regarding experimental models of vaginal candidiasis can be found in Cassone & Sobel (2016), Experimental Models of Vaginal Candidiasis and Their Relevance to Human Candidiasis, Infection and Immunity, 84(5):1255, which is incorporated by reference herein in its entirety.

Dosages of killer toxins as disclosed herein, or of organisms that express killer toxins, will typically be in the range of from greater than 0 mg/kg/day to 1 g/kg/day or more, such as 0.0001 mg/kg/day, 0.001 mg/kg/day, 0.01 mg/kg/day, 0.1 mg/kg/day, 1 mg/kg/day, 10 mg/kg/day, 100 mg/kg/day, 1 g/kg/day, or more. More typically, the dosage (or effective amount) may range from about 0.01 mg/kg to about 1 g/kg administered at least once per day, such as from 0.05 mg/kg to about 500 mg/kg or from about 0.1 mg/kg to about 750 mg/kg. A total daily dosage may range from about 1 mg/kg to about 250 mg/kg or to about 750 mg/kg per day, such as from 10 mg/kg to about 600 mg/kg per day or from about 25 mg/kg per day to about 800 mg/kg/day. Dosage amounts can be higher or lower depending upon, among other factors, the activity of the one or more organism (such as an S. cerevisiae strain expressing one or more killer toxins or an S. paradoxus strain expressing one or more killer toxins) and/or the one or more killer toxin (such as one or more K1, K2, and/or K1L toxins) administered, and/or its bioavailability, the mode of administration, and/or various other factors discussed herein.

Dosage amount and dosage interval can be adjusted for individual subjects in order to provide one or more organisms (such as an S. cerevisiae strain expressing one or more killer toxins or an S. paradoxus strain expressing one or more killer toxins) and/or one or more killer toxins (such as one or more, K2, and/or K1L toxins) in amounts sufficient to maintain therapeutic or prophylactic effect. For example, one or more organisms (such as an S. cerevisiae strain expressing one or more killer toxins or an S. paradoxus strain expressing one or more killer toxins) and/or one or more killer toxins (such as one or more K1, K2, and/or K1L toxins) can be administered once per day, multiple times per day, once per week, multiple times per week (such as every other day), one per month, multiple times per month, or once per year, depending upon, amongst other things, the mode of administration, the specific condition or conditions (such as a VVC, such as a VVC caused by C. glabrata) being treated, the susceptibility of the subject to the condition, and/or the judgment of the prescribing physician. One of ordinary skill in the art will be able to optimize effective local dosages without undue experimentation.

Compositions comprising one or more of the disclosed organisms (such as an S. cerevisiae strain expressing one or more killer toxins or an S. paradoxus strain expressing one or more killer toxins) and/or one or more of the disclosed killer toxins (such as one or more K1, K2, and/or K1L toxins), and/or one or more of the disclosed nucleic acid molecules, vectors, and/or host cells comprising one or more killer toxins typically comprise from greater than 0 up to 99% of the composition, which may further include one or more pharmaceutical acceptable excipients (such as, but not limited to, one or more buffers, carriers, salts, and/or therapeutic agents), by total weight percent. In specific, non-limiting examples, compositions comprising one or more of the disclosed organisms, killer toxins, nucleic acid molecules, vectors, and/or host cells comprise from about 1 to about 20 total weight percent of the one or more organisms, killer toxins, nucleic acid molecules, vectors, and/or host cells, and from about 80 to about 99 weight percent of a pharmaceutically acceptable additive.

Preferably, the one or more organisms (such as an S. cerevisiae strain expressing one or more killer toxins or an S. paradoxus strain expressing one or more killer toxins), one or more killer toxins (such as one or more K1, K2, and/or K1L toxins), and/or one or more of the disclosed nucleic acid molecules, vectors, and/or host cells comprising one or more killer toxins, and/or compositions thereof, will provide therapeutic and/or prophylactic benefit without causing substantial toxicity. Toxicity of organisms and/or killer toxins can be determined using standard pharmaceutical procedures. The dose ratio between toxic and therapeutic (or prophylactic) effect is the therapeutic index. Organisms (such as S. cerevisiae strains or S. paradoxus strains) and/or killer toxins (such as one or more, K2, and/or K1L toxins) that exhibit high therapeutic indices are preferred.

### V. Examples

The following examples are provided to illustrate certain features and/or embodiments of the disclosure. These examples should not be construed to limit the disclosure to the particular features or embodiments described. Changes therein and other uses that are encompassed within the spirit of the invention as defined by the scope of the claims will occur to those of ordinary skill in the art.

### Example 1

### Candida glabrata is Uniquely Susceptible to Antifungal Killer Toxins Expressed by Saccharomyces Yeasts

This example describes antifungal proteins (also known as “killer toxins”) expressed by the yeast Saccharomyces cerevisiae that were shown herein to inhibit the growth of more than 130 clinical and environmental isolates of the human pathogenic yeast Candida glabrata. The susceptibility of C. glabrata is unique amongst the yeasts of the Nakaseomyces clade and other pathogenic species of Candida (including the pathogenic yeast C. albicans and C. auris). The canonical killer toxins K1 and K2 are the most effective at inhibiting the growth of C. glabrata, inhibiting 100% and 98%, respectively, of all C. glabrata strains tested.

### Results

Saccharomyces Yeasts that Express Killer Toxins can Inhibit the Growth of Human Pathogenic Yeasts.

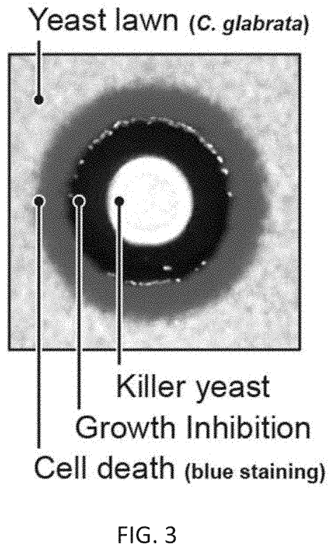

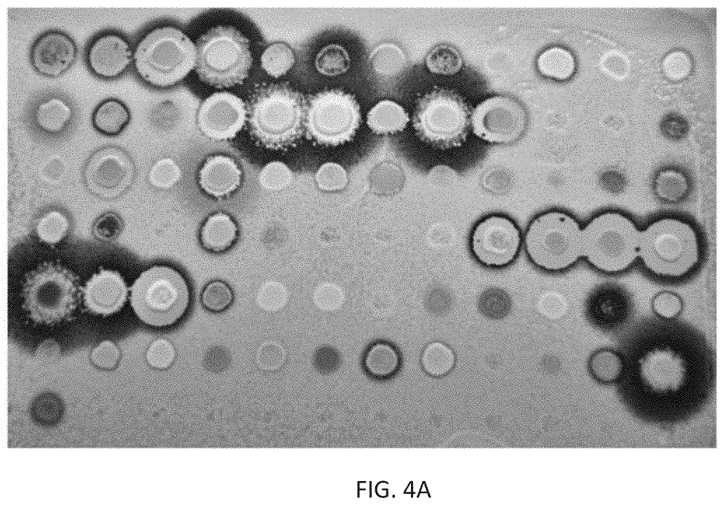

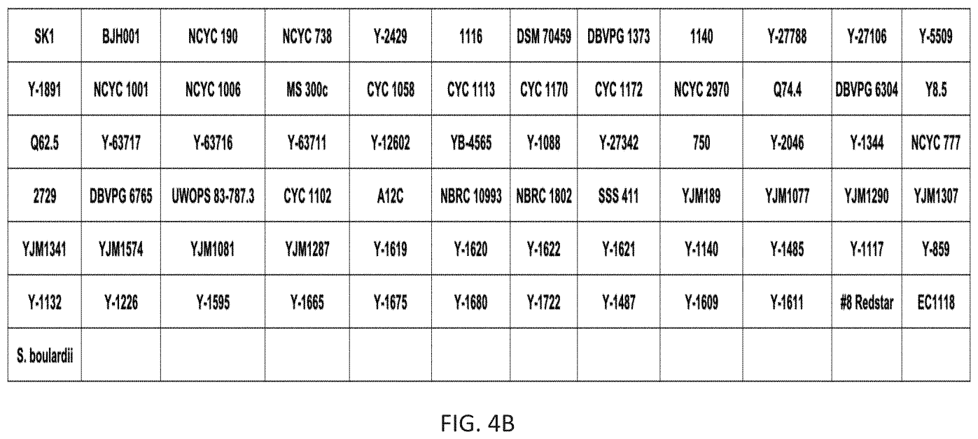

The most frequently used method to identify and quantify killer toxin production in yeasts is the “killer toxin assay.” Large scale inhibition assays can compete two strains of yeast on a pH 4.6 buffered YPD plate containing methylene blue as an indicator of cell death. Putative killer toxin-expressing yeasts are grown separately to high densities in liquid YPD in a 96 well plate prior to conducting the assay. The susceptible lawn strain is grown overnight then diluted 1:1,000 into water before being spread over a pH 4.6 methylene blue containing YPD plate. Once the low-density lawn suspension has dried on the surface of the YPD agar, a replicator is used to imprint as many as 96 concentrated suspensions of killer toxin-expressing yeasts (from the YPD cultures) on the seeded agar plate. This imprinting inoculates the agar plate with a concentrated drop of putative toxin producing yeast over the top of the lawn strain to allow for competition between yeasts. The production of a killer toxin that is inhibitory to the yeast of the yeast lawn can be determined by the presence of a zone of growth inhibition and/or a blue halo of oxidized methylene blue surrounding the killer toxin-expressing yeast colony (FIG. 3A). High-throughput robotics were used to screen a collection of approximately 1500 strains of Saccharomyces yeasts from various strain collections for killer toxin production (ARS Culture Collection (NRRL), National Collection of Yeast Cultures, and CBS-KNAW culture collection). Using this in vitro killer toxin assay, more than 200 Saccharomyces yeasts that express killer toxins and that are effective at inhibiting six different indicator strains of yeast from the Saccharomycetaceae were successfully identified.

Killer toxins have shown activity against certain human pathogenic fungi. More than 76 yeasts were identified that expressed killer toxins that could inhibit growth of the yeast S. cerevisiae (FIG. 14). To assess the antifungal activity of the identified collection of Saccharomyces yeasts that express killer toxins, a subset was used to challenge different Candida yeast species, including the important human pathogens Candida albicans, Candida glabrata, and Candida auris (Table 3). Of the pathogens screened, the type strain of C. glabrata (ATCC2001) was the most susceptible to killer toxins expressed by Saccharomyces yeasts, with 51% of the killer toxin-expressing yeasts assayed inhibiting C. glabrata (ATCC2001) growth. Challenging C. glabrata and four other Candida yeasts (C. albicans, C. rugosa, C. auris, and C. tropicalis) with nine canonical killer toxin-expressing strains demonstrated that the type strain of C. glabrata (ATCC_2001) is sensitive to the known killer toxins K1, K2, K21, K28, K45, K74, Y8.5, and K1us (FIG. 1). Although other Candida yeasts were susceptible to a subset of these toxins, C. glabrata was susceptible to a greater number of killer toxins than any other pathogenic yeast assayed.

TABLE 3
The type strain of C. glabrata is more susceptible to a greater
percentage of killer toxin-expressing Saccharomyces yeasts
than any other type-strain of pathogenic fungus tested.

| | Killer yeasts |
|---|---|
| Species | that inhibit growth (%) |
| Candida glabrata | 51 |
| Candida rugosa | 36 |
| Clavispora lusitaniae | 6 |
| Candida auris | 1 |
| Candida albicans | 0 |
| Candida parapsilosis | 0 |
| Candida railensis | 0 |
| Candida tropicalis | 0 |
| Pichia kudriavzevii | 0 |
| Cryptocaccus carnescens | 0 |
| Cryptococcus tephrensis | 0 |
| Cryptocaccus. victoriae | 0 |

Prior studies that demonstrated growth inhibition of C. glabrata by killer toxin-expressing yeasts assayed only a limited number of C. glabrata isolates. In the present study, a comprehensive collection of 135 C. glabrata strains was obtained from the collections of the American Type Culture Collection (ATCC), the Northern Regional Research Laboratory (NRRL, also referred to as the Agricultural Research Services (ARS) Culture Collection), FDA, CDC, Sobel Laboratory, Gabaldon laboratory, and the National collection of yeast cultures (NCYC) (FIG. 5, FIG. 13). These strains represent clinical and environmental C. glabrata isolates and encompass all currently known subpopulations of the species as determined by whole genome sequencing. Despite the known strain-specificity of killer toxins expressed by Saccharomyces yeasts, the present study found that 17 tested strains of S. cerevisiae yeasts that express killer toxins (strains YJM1574, YJM1081, YJM189, YJM1341, YJM1077, YJM1290, YJM1307, YO1620, YO1621, YO1622, EC1118, CYC1172, NCYC738, NCYC190, CYC1113, CYC1058 and BJH001) were broadly inhibitory to C. glabrata and were capable of inhibiting the growth of more than 98% of all C. glabrata strains challenged (FIG. 5).

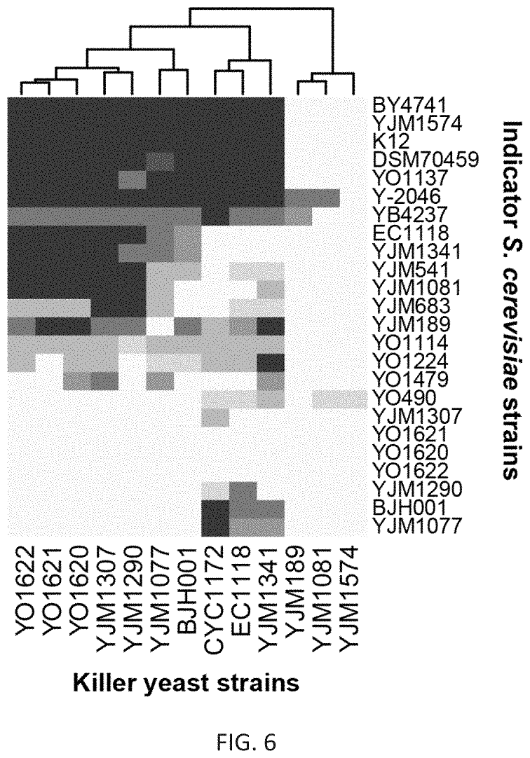

Many of the C. glabrata strains found susceptible to the tested killer toxins are known to be resistant to clinically relevant antifungal drugs, including azoles and echinocandins. To test whether the broad antifungal activity of the killer toxins expressed by 13 of the killer toxin-expressing yeast strains (YJM1574, YJM1081, YJM189, YJM1341, YJM1077, YJM1290, YJM1307, YO1620, YO1621, YO1622, EC1118, CYC1172, and BJH001) was specific to C. glabrata, 24 S. cerevisiae yeast strains were also assayed for killer toxin susceptibility (FIG. 6). Susceptibility to killer toxins varied between the different S. cerevisiae strains and all were resistant to at least one killer toxin. This demonstrated the strain-specific toxicity of these killer toxins towards S. cerevisiae, despite broad activity of the toxins against C. glabrata (FIG. 6). C. glabrata is classified within the Nakaseomyces and is more closely related to S. cerevisiae than to other Candida yeasts within the CUG clade (which includes all Candida species commonly isolated from human patients other than C. krusei and C. glabrata), with significant gene homology and synteny.

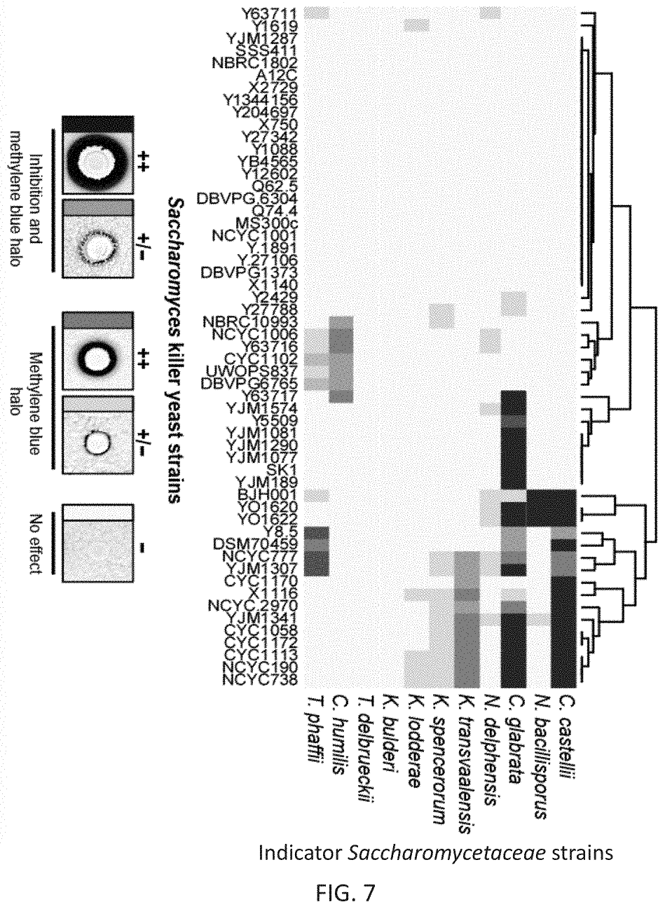

To test whether the acute susceptibility of C. glabrata to killer toxins extends to other Nakaseomyces yeasts and the family Saccharomycetaceae, 11 yeast species, including Nakaseomyces spp., Kazachstania spp., Naumovozyma spp., Tetrapisispora sp., and Torulaspora sp., were challenged by 58 different killer toxin-expressing yeasts, including the 13 killer toxin-expressing yeasts that appeared most inhibitory to C. glabrata. Most of the Saccharomycetaceae yeasts were resistant to killer toxins. C. glabrata remained the most susceptible species and was inhibited by 41% ( 24/58) of the killer toxin-expressing yeasts tested (FIG. 7). The next most susceptible yeast species was C. castellii, an environmental Nakaseomyces yeast that was inhibited by 28% ( 16/58) of the killer toxin-expressing yeasts tested (FIG. 7). Nakaseomyces species that were more resistant to killer toxins than C. glabrata also included the environmental yeasts N. delphensis and N. bacillisporus. Overall, these data showed that closely related species within the Nakaseomyces genus are significantly more resistant to killer toxins than C. glabrata. Due to the strain- and species-specificity of killer toxins, susceptibility to these toxins has been used as a measure of yeast diversity, to biotype different strains and species of pathogenic yeasts, and to identify different groups of unknown killer toxins. S. cerevisiae and C. glabrata exhibit high genetic similarity and similar genomic organizations, and both belong to the taxonomic group Saccharomycetaceae. Therefore, in the present study, susceptibility of different S. cerevisiae strains was used to identify different types of killer toxins that were also likely to have biological activity against C. glabrata. Clustering of the mosaic pattern of killer toxin susceptibilities observed in different S. cerevisiae strains suggested that two groups of killer toxins were most inhibitory to C. glabrata (FIG. 6). S. cerevisiae strain BJH001 is a K1 toxin producer that clusters with strains YJM1077, YJM1290, YJM1307, YO1620, YO1621, and YO1622 (group 1). All seven strains within this group demonstrate cross-immunity, strongly suggesting that the killer toxins produced by these strains are similar to K1. Killer toxin-expressing yeast strains YJM1290, YJM1307, YO1620, YO1621, and YO1622 have increased potency and a broader strain tropism against S. cerevisiae than canonical K1. The killer toxins expressed by the group 2 strains EC1118, CYC1172, and YJM1341 have another unique strain tropism and can inhibit growth of most S. cerevisiae strains. Further, EC1118, CYC1172, and YJM1341 were each immune to all group 2 toxins and were susceptible to group 1 (K1) toxins. Genetic sequencing of the dsRNAs within CYC1172 and YJM1341 suggests that the toxin expressed by these strains are closely related to K2. Thus, EC1118 is also likely to be a K2 killer toxin-expressing yeast. It appears that despite the closer evolutionary relationship to C. glabrata, many S. cerevisiae strains are nonetheless resistant to the 13 killer toxins that are the most inhibitory to C. glabrata growth (FIG. 6).

Satellite dsRNA-Encoded K1 and K2 Killer Toxins are Most Inhibitory to Candida glabrata.

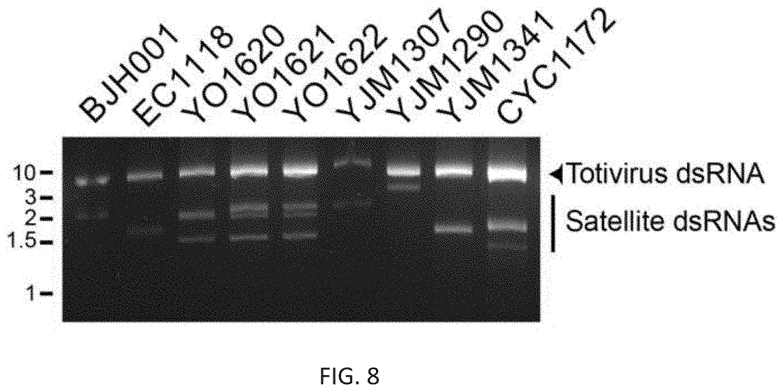

Killer toxins expressed by Saccharomyces yeasts are often encoded by one or more dsRNA satellites of viruses, such as totiviruses, present in the yeasts. Such dsRNA satellites encode a variety of killer toxins in multiple species of fungi. In the present study, 13 S. cerevisiae strains that were most inhibitory to C. glabrata were assayed for the presence of dsRNAs using cellulose chromatography. Ten strains comprised dsRNAs with molecular weights associated with totiviruses and satellite dsRNAs (FIG. 8). Exposure of these strains to cycloheximide cured these yeasts of the dsRNA satellites and resulted in loss of killer toxin production. This indicated that the killer toxins responsible for C. glabrata inhibition were likely encoded by satellite dsRNAs. To determine the genetic sequence of these killer toxins, dsRNAs were extracted and purified from representative groups 1 and 2 strains (YO1622, YJM1077, YJM1307, YJM1341, and CYC1172) and their genetic sequences were determined using next-generation short-read sequencing as described in Crabtree A M et al, Viruses, 2019, 11:70. As expected from their strain tropism, analysis of the assembled high quality contigs identified that group 1 killer toxin-expressing yeasts encoded K1-type toxins, and group 2 encoded K2-type toxins. These killer toxins were approximately 98% identical at the amino acid level to the canonical killer toxins previously described in the literature. To more quantitatively assess the contribution of polymorphisms within K1 and K2 toxins to their strain tropism and potency, dsRNAs were cloned from the K1-expressing strains YJM1307 and YO1622, and from the K2-expressing strains CYC1172 and YJM1341 by rt-PCR into yeast plasmid vectors and ectopically expressed in the yeast strain BY4741, which otherwise does not express killer toxins. These cloned toxins were all active and inhibited S. cerevisiae.

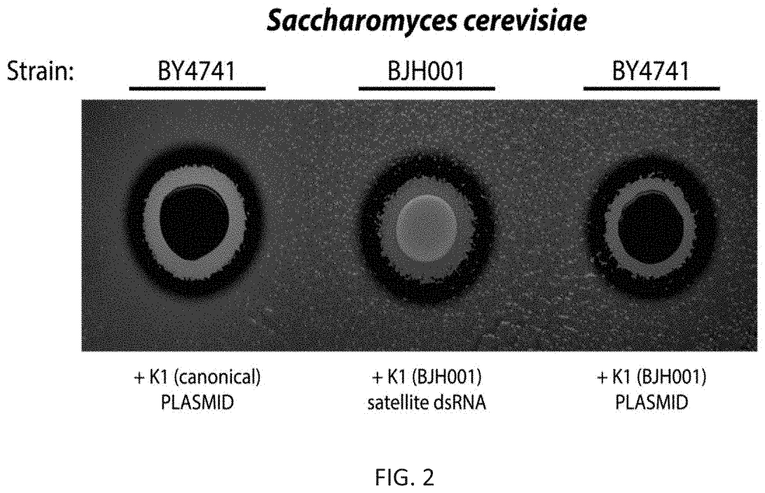

To independently confirm that antifungal activity against C. glabrata resulted from S. cerevisiae expression of killer toxins, the K1 toxin was cloned and expressed in the non-killer strain of Saccharomyces cerevisiae strain BY4741 (FIG. 2). Expression of the canonical K1 or a K1 variant previously described in Crabtree A M et al., Viruses, 2019, 11:70 was as effective at inhibiting the growth of C. glabrata as was endogenous K1 expression from a satellite dsRNA by S. cerevisiae strain BJH001 (FIG. 2).

### Example 2

### K1 and K2 Toxins are Active Against C. glabrata Under Vaginal-Simulative Conditions

C. glabrata is thought to be a major cause of persistent and drug resistant VVC and is often isolated from the vaginal mucosa. Example 1 showed that killer toxins appear to be uniquely suited for combatting VVC based on their broad spectrum of activity against C. glabrata (as described above). The killer toxins identified in the present studies are active at the pH of the vaginal mucosa (pH 4.6) (FIG. 9), while topical therapeutics, such as Amphotericin B and azoles, are less effective at this pH.

The optimum pH for each of the toxins expressed by the killer toxin-expressing yeasts shown in FIG. 9 was determined using the agar plate killer toxin assay as described previously in Example 1. Briefly, a concentrated suspension of killer toxin-expressing yeasts (strains NCYC190, BJH001, CYC1058, CYC1113, CYC1172, DBVPG6304, NCYC1001, NCYC738, NCYC777, SK1, Y-1088, Y-12602, Y-2429, Y-27432, Y-63711, Y-63716, Y-63717, Y8.5, and YB-4565 in FIG. 9) was inoculated onto a YPD agar plate (pH 4.6) with methylene blue. A low-density lawn of an indicator strain of yeast (S. cerevisiae BY4741 in FIG. 9) was spread and dried on the surface of the plate before the killer toxin-expressing yeast strain was inoculated. The production of a killer toxin that is inhibitory to the yeast of the yeast lawn can be determined by the presence of a zone of growth inhibition and/or a blue halo of oxidized methylene blue surrounding the killer toxin-expressing yeast colony (See FIG. 3). However, the agar plates were pH-balanced to a range of different pH values. The optimum pH for each toxin was identified as the pH that allowed for formation of the largest zone of growth inhibition (indicated by substantially 100% inhibition). All other zones of inhibition were measured and their relative sizes were scaled as a percentage.

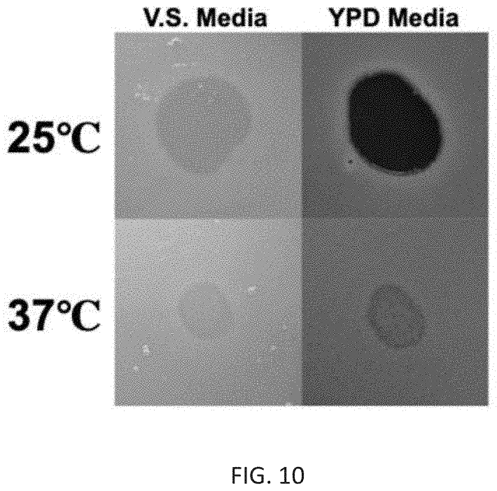

Toxin activity was tested under more realistic conditions using vaginal-simulative media and a hypoxic atmosphere. Vaginal-simulative media simulates the chemical environment of the vaginal mucosa. The modified (hypoxic) atmosphere further simulates conditions within the vagina. Hypoxic conditions in this Example comprised 2% oxygen, 5% CO₂, and 93% nitrogen. A K1 toxin expressed by S. cerevisiae strain YJM1307 was purified using either ethanol or ammonium sulfate precipitation. The same procedures were used to purify a K2 killer toxin from strain CYC1172; however, the purification using ethanol failed to yield a concentrated K2 toxin. Under the vaginal-simulative conditions described above in this Example, there was no loss of killer toxin activity against C. glabrata (ATCC2001) (FIG. 10).

### Example 3

### Ethanol Precipitation as an Exemplary Method for Purifying Killer Toxins from Cell Cultures

This example describes an exemplary method of separating K1 and/or K2 toxins from fungi and fungal cultures for application against pathogenic fungi. The example shows that ethanol precipitation is an effective alternative to ammonium sulfate for the purification of active K1 toxin but is not suitable for the purification of active K2.

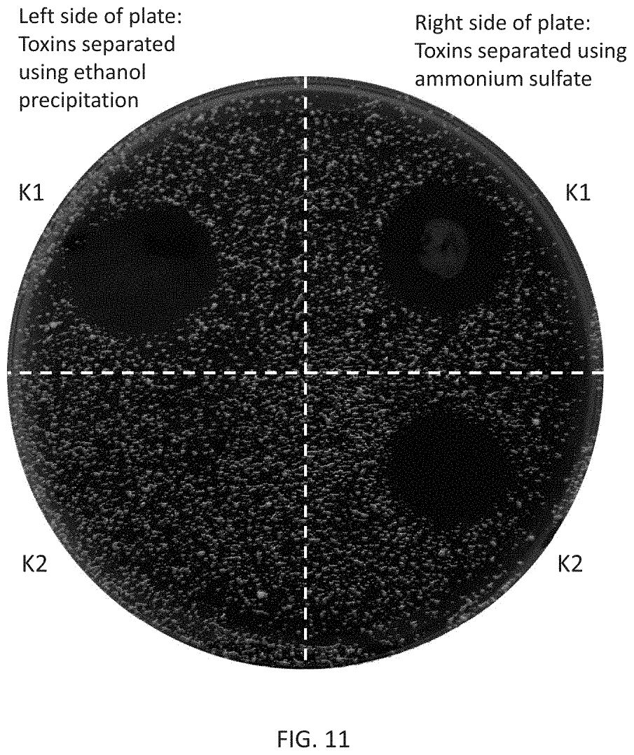

K1 and K2 expressing strains of S. cerevisiae were grown for 24 hours at 25° C. in pH 4.6 YPD medium. Culture supernatant was collected and mixed 1:1 with supersaturated ammonium sulfate or 100% ethanol before being placed at 4° C. for 3 hours. Tubes containing the mixtures were centrifuged at 21,000 RCF for 10 minutes. The supernatant was removed, and the tubes were washed with pH 4.6 YPD. The wash suspension was then plated on a BY4741 S. cerevisiae lawn to test biological activity of the separated toxins. FIG. 11 shows biological activity of the K1 toxin against the BY4741 yeast strain following separation using either ethanol or ammonium sulfate, and biological activity of the K2 toxin following purification using ammonium sulfate, but no activity of the K2 toxin following purification using ethanol.

### Example 4

### Stability of K1 and K2 Toxins at 37° C.

To ascertain the stability of killer toxins at human body temperature, purified killer toxins from yeast cultures were incubated at 37° C. for up to 1 hour and 45 minutes. This example shows that the K2 toxin is heat stable for at least the duration of the assay (1 hour and 45 minutes). While the K1 toxin showed no activity at the 30-minute timepoint, yeast extract stabilized both toxins.

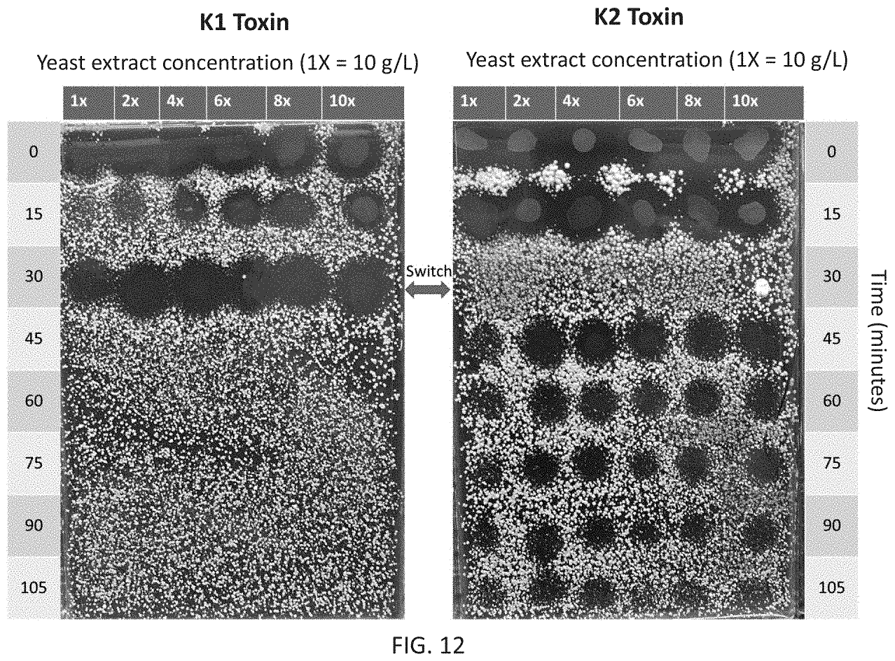

K1 and K2 expressing strains of S. cerevisiae were grown for 24 hours at 25° C. in pH 4.6 YPD. The toxin was then precipitated with supersaturated ammonium sulfate and resuspended in 1× (10 g/L), 2× (20 g/L), 4× (40 g/L), 6× (60 g/L), 8× (80 g/L), or 10× (100 g/L) pH 4.6 yeast extract. The resuspended tubes were then incubated at 37° C. for 1 hour and 45 minutes. Five μL was removed and plated to test for activity every 15 minutes. The test lawn strain was S. cerevisiae BY4741 on pH 4.6 YPD stained with methylene blue. Assay results showed that the K2 toxin maintains killing ability through all time points (FIG. 12). The K1 toxin was inactivated after 30 min incubation at 37° C. Compared to a simple buffer alone where both toxins were inactive (sodium citrate pH 4.6), the addition of (Fisher Scientific brand) yeast extract (pH 4.6) as an adjuvant stabilized both killer toxins when added at a concentration above 10 g/L. In addition, K1 and K2 appear to be stabilized further by increasing concentrations of yeast extract above 10 g/L, with maximum stabilization at 60 g/L and 20 g/L, respectively (FIG. 12). Such stabilization is exemplified in FIG. 12 by the larger zones of inhibition at 20 g/L yeast extract than 10 g/L yeast extract, especially after 15 min for K1 and after 45 min for K2. Other tests for effective adjuvants have found that dextrose can also stabilize K1.

### Example 5

### K1 Killer Toxin Stabilization Using Yeast Extract

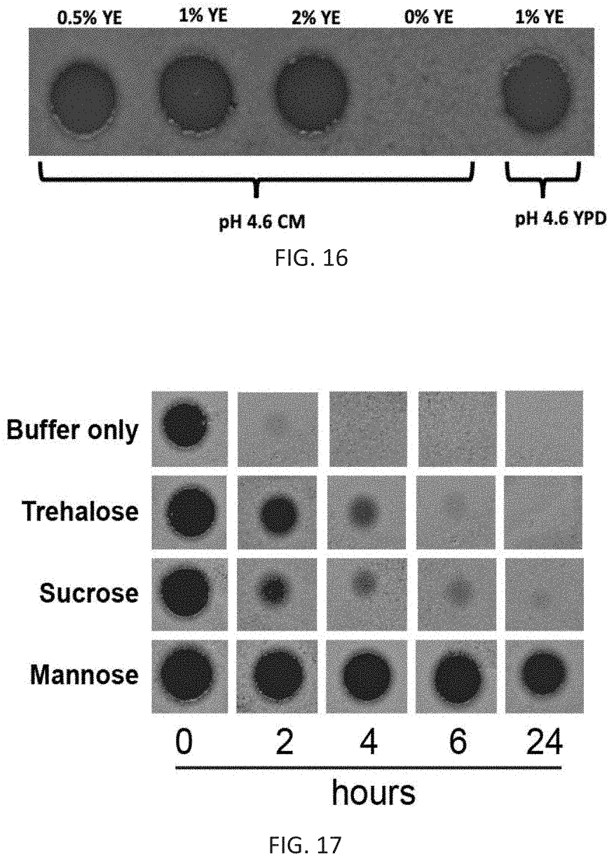

K1 killer toxin cannot be purified by precipitation from complete liquid media (CM) containing all 20 amino acids, ammonium sulphate, and dextrose unless the media is complemented with yeast extract (FIG. 16, ‘0% YE’). The present example shows the effect of three different concentrations of yeast extract (0.5%, 1%, and 2%) on stabilization of K1 toxin in CM with 2% dextrose.

CM containing 2% dextrose and 0.5%, 1%, or 2% yeast extract was used to grow the K1 toxin-producing strain Saccharomyces cerevisiae YJM1307 at room temperature for three days before ethanol precipitation. The protein pellet was suspended in 0.5% yeast extract at pH 4.6 and tested for activity against Candida glabrata ATCC2001 (FIG. 16). These results showed that yeast extract is required for efficient K1 toxin production in CM media.

### Example 6

### K1 Killer Toxin Stabilization Using Carbohydrates

K1 toxin loses its antifungal activity rapidly at 37° C. when the purified toxin is suspended in a simple buffer at pH 4.6 (2.9% w/v sodium citrate in deionized H₂O) (FIG. 17, top row). The present example shows for the first time that K1 can be stabilized at 37° C. using mannose, trehalose, sucrose.

Glycerol and gelatin have been shown to stabilize the K2 toxin at 22° C. However, neither of these compounds stabilized the K1 toxin in the present work. Yeast extract has been shown to improve the activity of K1 and other killer toxins (Examples 4-5). Yeast extracts contain cell wall carbohydrates from yeasts, including cell wall mannans (complex mannose polymers). Mannans were tested and found to stabilize K1 in the present studies.

After K1 precipitation from CM, different types and concentrations of carbohydrates were assayed for their ability to stabilize K1 at 37° C. Mannose (which is less expensive and more readily available than mannans, e.g., D-(+)-mannose >99% from Fisher Scientific (AC150600250)), trehalose, and sucrose all improved K1 toxin stability at 37° C. at 30% w/v in a pH 4.6 buffered sodium citrate solution (the pH of this solution was adjusted to 4.6 with HCl before the solution was filtered for sterility). Two hundred microliters of each buffered carbohydrate solution were then added to 13 ng of K1 killer toxin, and 3.9 μL (2.6 ng) were used to treat a C. glabrata (ATCC2001) lawn on a YPD agar plate buffered to pH 4.6 with methylene blue. The remaining toxin suspension was incubated at 37° C. for 2, 4, 6, and 24 hours, and 3.9 μL were used to treat a C. glabrata (ATCC2001) lawn at each timepoint (FIG. 17). These results showed that mannose, trehalose, and sucrose stabilize K1 at 37° C. compared to K1 that is suspended in a simple buffer. K1 toxin stabilized with mannose showed activity even after 24 hours. In some embodiments, sucralose may be used.

### Example 7

### Optimization of K1 Production Using Yeast Fermentation

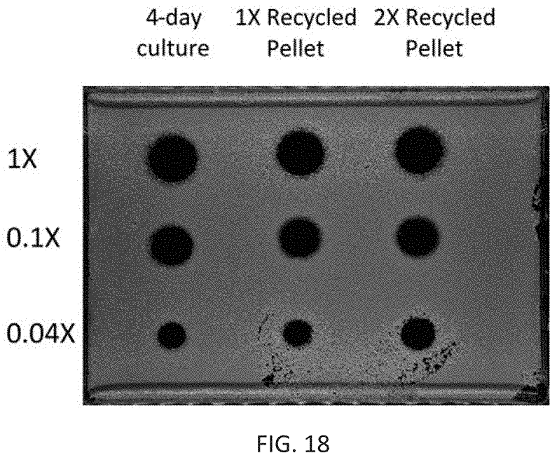

To increase the quantity of K1 toxin obtainable from cultured cells, a protocol was developed to extract K1 toxin from cells over three days. A 25-mL flask of pH 4.6 YPD inoculated with S. cerevisiae YJM1307 was grown for four days. The culture medium was then separated from the yeast and K1 toxin was precipitated from the medium using ethanol (FIG. 18, ‘4-day culture’). Harvested yeast cells were then suspended in 25 mL of fresh YPD (pH 4.6) and incubated for another 24 hours. The resulting culture medium was again separated and K1 was precipitated using ethanol (FIG. 18, ‘1× Recycled Pellet). The process of resuspending the yeast cells, growing for another 24 hours, and precipitating K1 toxin from the culture medium using ethanol was repeated a second time (FIG. 18, ‘2× Recycled Pellet). At each stage of toxin harvesting, the precipitated K1 toxin was suspended in 0.5% yeast extract and diluted (1×, 0.1× and 0.04×) to show the relative quantity of toxin at each of the three different stages. These results showed that toxin yield was consistent at each stage.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A composition, comprising:
at least one killer toxin, an organism that expresses the at least one killer toxin, or the at least one killer toxin and the organism that expresses the at least one killer toxin, wherein the at least one killer toxin comprises a K1, K2, K28, K1us, K74, K21, K62, K45, K1L, KHR, or KHS protein, or a combination thereof; and
one or more pharmaceutically acceptable excipients selected from the group comprising an adjuvant, a carrier, a buffer, and a combination thereof.

2. The composition of claim 1, wherein the organism is a yeast, a recombinant yeast, a recombinant bacterium, a recombinant animal cell, a recombinant virus, or a combination thereof.

3. The composition of claim 2, wherein the yeast is a member of genus Saccharomyces.

4. The composition of claim 1, wherein
the at least one killer toxin comprises the K1 protein and the K1 protein comprises an amino acid sequence at least 80% identical to any one of SEQ ID NOs: 1-12;
the at least one killer toxin comprises the K2 protein and the K2 protein comprises an amino acid sequence at least 80% identical to any one of SEQ ID NOs: 13-18; or
the at least one killer toxin comprises the K1L protein and the K1L protein comprises an amino acid sequence at least 80% identical to SEQ ID NO: 38.

5. The composition of claim 1, wherein the composition is an ointment, cream, suppository, capsule, ovule, tablet, pill, solution, suspension, foam, film, gel, liposomal composition, or a combination thereof.

6. A vector comprising the at least one killer toxin of claim 1.

7. An isolated host cell comprising the vector of claim 6.

8. A method for treating and/or preventing a yeast infection in a subject, the method comprising administering to the subject the composition of claim 1.

9. The method of claim 8, wherein the subject is a human.

10. The method of claim 8, wherein the yeast infection is caused by Candida glabrata.

11. The method of claim 8, wherein the yeast infection is vulvovaginal candidiasis.

12. A method for treating and/or preventing a yeast infection caused by Candida glabrata in a subject, the method comprising administering to the subject:
at least one killer toxin;
at least one organism that expresses the at least one killer toxin; or
a combination thereof.

13. The method of claim 12, wherein the subject is a human.

14. The method of claim 12, wherein the organism is a yeast, a recombinant yeast, a recombinant bacterium, a recombinant animal cell, a recombinant virus, or a combination thereof.

15. The method of claim 14, wherein the yeast or the recombinant yeast is a member of genus Saccharomyces.

16. The method of claim 12, wherein the yeast infection is vulvovaginal candidiasis.

17. The method of claim 12, wherein the at least one killer toxin comprises a K1, K2, K28, K1us, K74, K21, K62, K45, K1L, KHR, or KHS protein, or a combination thereof.

18. The method of claim 17, wherein
the at least one killer toxin comprises the K1 protein and the K1 protein comprises an amino acid sequence at least 80% identical to any one of SEQ ID NOs: 1-12;
the at least one killer toxin comprises the K2 protein and the K2 protein comprises an amino acid sequence at least 80% identical to any one of SEQ ID NOs: 13-18; or
the at least one killer toxin comprises the K1L protein and the K1L protein comprises an amino acid sequence at least 80% identical to SEQ ID NO: 38.

19. The method of claim 12, further comprising administering to the subject one or more pharmaceutically acceptable excipients selected from the group comprising an adjuvant, a carrier, a buffer, and combinations thereof.

20. A kit, comprising
at least one killer toxin;
an organism that expresses the at least one killer toxin;
an applicator selected from the group comprising a tampon, a vaginal ring, a pillow, a puff, a sponge, or an osmotic pump system;
an applicator useful for administering a cream and/or ointment;
an applicator useful for intravaginal administration of a capsule, ovule, tablet, pill, and/or suppository; or
combinations thereof.


SEQUENCE LISTING (38 sequences)

SEQ ID NO: 1 - protein, 234 aa, Saccharomyces cerevisiae
GVVIFGINVCKNCVGERKDDSSTDCGKQTLALLVSIFVAVTSGHYLIWGGNRPVSQSDPNGATVARRDISTVADGDIPLDFSALNDILNEHGISILPANASQYVKRSDTAKHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 2 - protein, 306 aa, Saccharomyces cerevisiae
SVSILFFITLLHLVVALNDVASPAEIAPVSLLPREAPWYDKIWEVKDWLLQRATDGNWGKSITWGSFVASDAGVVIFGINVCKNCVGERKDDSSTDCGKQTLALLVSIFVAVTSGHYLIWGGNRPVSQSDPNGATVARRDISTVADGDIPLDFSALNDILNEHGISILPANASQYVKRSDTAKHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 3 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 4 - protein, 309 aa, Saccharomyces cerevisiae
SSRGLGDVYKRQLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHTPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 5 - protein, 300 aa, Saccharomyces cerevisiae
YQRLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGATVARRDISTVADGDIPLVFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 6 - protein, 215 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGATVARRDISTVADGDIPLDFSALNDILNEHGISILPANASQYVKRSDTAEHTTSFCSDQQLHFFAYRPDSSW

SEQ ID NO: 7 - protein, 330 aa, Saccharomyces cerevisiae
KDDSSMDCGKQTLALLVSIFVAVTSVSILFFITLLHLVVALNDVAGPAETAPVSLLPREAPWYDKIWEVKDWLLQRATDGNWGKSITWGSFVASDAGVVIFGINVCKNCVGERKDDSSMDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 8 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDTGVVIFGINVCKNCVGERKDDGGTDCGKQTLALLVSIFVAVTSGHHLIWGGNRPVSPSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGY

SEQ ID NO: 9 - protein, 296 aa, Saccharomyces cerevisiae
LHLVVALNDVAGPAETAPVSLLPREAPWYDKIWEVKDWLLQRATDGNWGKSITWGSFVASDTGVVIFGINVCKNCVGERKDDGGTDCGKQTLALLVSIFVAVTSGHHLIWGGNRPVSPSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGY

SEQ ID NO: 10 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGAIVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 11 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDSSTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMAFNDAMVSANGNLYGLAEKLFSEDEGQWETNYYKLYWSTGQWIMSMKFIEESIDNANNDFEGCDTGH

SEQ ID NO: 12 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDISTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 13 - protein, 362 aa, Saccharomyces cerevisiae
MKETTTSLMRDELTLGEPAAQARMCVRLLRFFIGLTITAFIIAACIIKSATGGSGYSNAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLSAAATAYKAYATNTTRVGKRQDGEWVSYSVYGENVDYERYPVAHLQEEADACYESLGNMITSQVQPCTQRECYAMDQKVCAAVGFSSDVGVNSAMVGEAYFYAYGGVDGECDSG

SEQ ID NO: 14 - protein, 354 aa, Saccharomyces cerevisiae
MQDELTLGEPATQARMCVRLLRFFIGLTITAFIIAACIIKSATGGSGYSNAVAVRGEADTPSTIVGQLVERGGFQAWAVGAGICLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYATNTTRVGKRQDSEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 15 - protein, 362 aa, Saccharomyces cerevisiae
MKETTTSLMQDELTLGEPATQARMCVRLLRFFIGLTITAFVIAACIIKSATGGSGYSNAVAVWGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYATNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 16 - protein, 362 aa, Saccharomyces cerevisiae
MKETTTSLMQDELTLGEPATRARMCVRLLRFFIGLTITAFIIAACIIKSATGGSGYSNAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSLLLNNTLYVGGDHTADYDSELATILGSVYNDVVHLGVYYDNSTGIVKRDSRPSMISWTVLHDNMMITSYHRPDQLGAAATAYKAYATNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 17 - protein, 362 aa, Saccharomyces cerevisiae
MKETTTSLMQDELTLGEPATQARMCVRLLRFFIGLTITAFIIAACIIKSATGGSGYSNAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYATNTTRVGKRQDGEWVSYSVYGENVDYERYPVAHLQEEADACYESLGNMITSQVQPCTQRECYAMDQKVCAAVGFSSDAGVNSAIVGEAYFYAYGGVDGECDSG

SEQ ID NO: 18 - protein, 362 aa, Saccharomyces cerevisiae
MKETTTSLMQDELTLGEPATQARMCVRLLRFFIGLTITAFIIAACIIKSATGGSGYSKAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYTTNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 19 - DNA, 853 bp, Saccharomyces cerevisiae
aggtgtagtaatctttggtatcaatgtgtgtaagaactgcgtgggtgagcgtaaggatgatagcagtacggactgcggcaagcaaacacttgctttactagtcagcatttttgtagcagttacatccggccattatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagccaacgcatcacaatatgtcaaaagatcagacacagccaaacacacgacaagttttgtagtgaccaacaactacacttctttgcataccgatctgattcatcatggtaatggaacatacaccacgtttaccacacctcacattccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactagggcatcgtgtctgacctctgatgcgataacttgaccctacagagcaccgggccatatataatatagtaggcctgtctcttatacacatctgacgctgccgacgagtgcaagagtgtagatctcggtggtcgccgtatcattaaaaaa

SEQ ID NO: 20 - DNA, 1131 bp, Saccharomyces cerevisiae
atccgtcagtatattatttttcatcacattactacacctagtcgtagcgctgaacgatgtggccagtcctgcagaaatagcaccagtgtcattactacctcgtgaagcgccgtggtatgacaagatctgggaggtaaaagattggctattacagcgtgccacagatggcaattggggcaagtcgatcacctggggttcattcgtagcaagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccattatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagccaacgcatcacaatatgtcaaaagatcagacacagccaaacacacgacaagttttgtagtgaccaacaactacacttctttgcataccgatctgattcatcatggtaatggaacatacaccacgtttaccacacctcacattccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactagggcatcgtgtctgacctctgatgcgataacttgaccctacagagcaccgggccatatataatctgtctcttatacacatctccgagcccacgagacccatcacaatctcgtatgccgtcttctgcttgaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - DNA, 951 bp, Saccharomyces cerevisiae
atgacgaagccaacccaagtattagttagatccgtcagtatattatttttcatcacattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacattccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactag

SEQ ID NO: 22 - DNA, 1109 bp, Saccharomyces cerevisiae
ttttttttcaagcagaagacggcatacgagatgaatgatcgtctcgtgggctcggagatgtgtataagagacagttactgcacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacactccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactagagcatcgtgtctgacctctgatgcgatatctcgaccctacagagcaccgggctatatataatatctgtctcttatacacatctccgagcccacgagacgatcattcatctcgtatgccgtcttctgcttgaaaaaaaaaaa

SEQ ID NO: 23 - DNA, 1135 bp, Saccharomyces cerevisiae
ttggctattacagcgtgccacagatggcaattggggcaagtcgatcacctggggttcattcgtagcgagcgatgcagttcttacacacattgataccaaagattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggtctttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacattccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactagggcatcgtgtctgacctctgatgcggtaactcgaccctacagagcaccgggctatatataatatctgtctcttatacacatctccgagcccacgagacgatcattcatctcgtatgccgtcttctgcttgaaaaaaaaa

SEQ ID NO: 24 - DNA, 952 bp, Saccharomyces cerevisiae
atgacgaagccaacccaagtattagttagatccgtcagtatattatttttcatcacattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggactttagtgcgttgaacgatatattaaatgaacatggtattagtatactcccagctaacgcatcacaatatgtcaaaagatcagacacagccgaacacacgacaagtttttgtagtgaccaacaactacacttctttgcataccgacctgattcatcatggtaatggaacatataccacgtttaccacacctcacactccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactag

SEQ ID NO: 25 - DNA, 1135 bp, Saccharomyces cerevisiae
tttttttttttcaagcagaagacggcatacgagattacgacctgtctcgtgggctcggagatgtgtataagagacagatattatatatagcccggtgctctgtagggtcgagttatcgcatcagaggtcagacacgatgccctagtggcctgtgtcacagccttcaaagtcattattggcgttatcaatactttcctcaataaacttcatcgacattatccactggccagtactccaatacaatttatagtaattcgtctcccattgtccctcatcctcactaaacagcttttctgctagtccatacaggttaccattagccgacaccatggcatcattaagggccatacagtatgaggccttgataccatgctcgcacataggataaacataacgcttggccactgctggaatgtgaggtgtggtaaacgtggtatatgttccattaccatgatgaatcaggtcggtatgcaaagaagtgtagttgttggtcactacaaaacttgtcgtgtgttcggctgtgtctgatcttttgacatattgtgatgcgttagctgggagtatactaataccatgttcatttaatatgtcgttcaacgcactaaagtccagtggaatatccccgtctgcgacagtagaaatgtcacgacgagcaacggtagcgccattaggatctgactgcgacaccggcctattaccaccccatataagatgatggccggatgtaactgctacaaaaatgctgactagtaaagcaagtgtttgcttgccgcagtccatactgctatcatccttacgctcacccacgcagttcttacacacattgataccaaagattactacacctgcatcgctcgctacgaatgaaccccaggtgatcgacttgccccaattgccatctgtggcacgctgtaatagccaatcttttacttcccagatcttgtcataccacggcgcttcacgaggtagtaatgacactggtgctgtttctgcaggaccggccacatcgttcagcgctacgactaggtgtagtaatgtgatgaaaaataatatactgacggatgtaactgctacaaaaatgctgactagtaaagcaagtgtttgcttgccgcagtccatactgctatcatcctta

SEQ ID NO: 26 - DNA, 951 bp, Saccharomyces cerevisiae
atgacgaagccaacccaagtattagttagatccgtcagtatattatttttcatcacattactacacctagtcgtagcgctcaacgatgtggccggtcctgcagaaacagcaccagtgtcattactacctcgtgaagcgccgtggtatgacaagatctgggaagtaaaagattggctattacagcgtgccacagatggcaattggggtaagtcgatcacctggggttcattcgtagcgagcgatacaggtgtagtaatctttggtatcaatgtatgtaagaactgcgtgggtgagcgtaaggatgatggcggtacggactgcggcaagcaaacacttgctttactagtcagcatttttgtagcagttacatccggccatcatctaatatggggtggtaataggccggtgtcgccgtcagatcctaatggcgctaccgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttactacacctcacattccagcagtggcgaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggctactag

SEQ ID NO: 27 - DNA, 1076 bp, Saccharomyces cerevisiae
gatttttattttattttattttattttgtgcctactatattatatatagcccggtactctgtagggtcgagttatcgcatcagaggtcagacacgatgccctagtagcctgtgtcacagccttcaaagtcattattggcgttatcaatactttcctcaataaacttcatcgacattatccactggccagtactccaatacaatttatagtaattcgtctcccattgtccctcatcctcactaaacagcttttctgctagtccatacaggttaccattagccgacaccatggcatcattaagggccatacagtatgaggccttgataccatgctcgcacataggataaacataacgcttcgccactgctggaatgtgaggtgtagtaaacgtggtatatgttccattaccatgatgaatcaggtcggtatgcaaagaagtgtagttgttggtcactacaaaacttgtcgtgtgttcggctgtgtctgatcttttgacatattgtgatgcgttagctgggagtatactaataccatgttcatttaatatgtcgttcaacgcactaaagtccagtggaatatccccgtctgcgacagtagaaatgtcacgacgagcaacggtagcgccattaggatctgacggcgacaccggcctattaccaccccatataagatgatggccggatgtaactgctacaaaaatgctgactagtaaagcaagtgtttgcttgccgcagtccgtaccgccatcatccttacgctcacccacgcagttcttacatacattgataccaaagattactacacctgtatcgctcgctacgaatgaaccccaggtgatcgacttgccccaattgccatctgtggcacgctgtaatagccaatcttttacttcccagatcttgtcataccacggcgcttcacgaggtagtaatgacactggtgctgtttctgcaggaccggccacatcgttgagcgctacgactaggtgtagtaatgtgactgtctcttatacacatctccgagcccacgagacgtagaggaatctcgtatgccgtcttctgcttgaaaaaaaaaaa

SEQ ID NO: 28 - DNA, 951 bp, Saccharomyces cerevisiae
atgacgaagccaacccaagtattagttagatccgtcagtatattatttttcatcacattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgcgttactagtcagcatttttgtagcagttacatccggccatcatcttatatggggtggtaataggccggtgtcgcagtcagatcctaatggcgctatcgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacattccagcagtggccaagcgttatgtttaccctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactag

SEQ ID NO: 29 - DNA, 951 bp, Saccharomyces cerevisiae
atgacgaagccaacccaagtattagttagatccgtcagtatattatttttcatcacattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatagcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacattccagcagtggccaagcgttatgtttatcctatgtgcgagcatggtatcaaggcctcatactgtatggcctttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactag

SEQ ID NO: 30 - DNA, 951 bp, Saccharomyces cerevisiae
atgacgaagccaacccaagtattagttagatccgtcagtatattatttttcatcacattactacacctagtcgtagcgctgaacgatgtggccggtcctgcagaaacagcaccagtgtcattactacctcgtgaagcgccgtggtatgacaagatctgggaagtaaaagattggctattacagcgtgccacagatggcaattggggcaagtcgatcacctggggttcattcgtagcgagcgatgcaggtgtagtaatctttggtatcaatgtgtgtaagaactgcgtgggtgagcgtaaggatgatatcagtacggactgcggcaagcaaacacttgctttactagtcagcatttttgtagcagttacatccggccatcatcttatatggggtggtaataggccggtgtcgcagtcagatcctaatggcgctaccgttgctcgtcgtgacatttctactgtcgcagacggggatattccactggactttagtgcgttgaacgacatattaaatgaacatggtattagtatactcccagctaacgcatcacaatatgtcaaaagatcagacacagccgaacacacgacaagttttgtagtgaccaacaactacacttctttgcataccgacctgattcatcatggtaatggaacatataccacgtttaccacacctcacattccagcagtggccaagcgttatgtttatcctatgtgcgagcatggtatcaaggcctcatactgtatggcccttaatgatgccatggtgtcggctaatggtaacctgtatggactagcagaaaagctgtttagtgaggatgagggacaatgggagacgaattactataaattgtattggagtactggccagtggataatgtcgatgaagtttattgaggaaagtattgataacgccaataatgactttgaaggctgtgacacaggccactag

SEQ ID NO: 31 - DNA, 1089 bp, Saccharomyces cerevisiae
atgaaagagactaccaccagcctgatgcgagacgagctgacactaggtgagccggccgcccaagcaaggatgtgcgtacgtctattacgttttttcataggtctgactataaccgcatttattatagcagcctgtattattaaaagtgcgacaggcggttcgggatattctaatgcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgagcgcagccgcgacagcctacaaagcttatgccacaaacacaacacgggtcggtaagaggcaggacggtgagtgggtgtcgtactcggtctacggtgagaatgttgactatgaaagataccctgtagcacatctgcaagaggaggccgacgcgtgttacgagagtttaggcaatatgattacgagccaggtacagccctgtactcagagagaatgttatgctatggatcagaaagtatgcgcagctgtcggcttctcatcagatgtgggtgttaactccgcaatggtcggtgaggcctacttctatgcctatggtggggttgatggtgaatgtgacagcggctag

SEQ ID NO: 32 - DNA, 1604 bp, Saccharomyces cerevisiae
gcctgatgcaagacgagctgacactaggtgagccggccacccaagcaaggatgtgcgtacgtctattacgttttttcataggtctgactataaccgcatttattatagcagcctgtattattaaaagtgcaacaggcggctcgggatattctaatgcagttgctgttcggggagaagcggacaccccttccacaattgtgggccagctcgtcgagcgtggcggcttccaagcttgggcagtgggggctggtatctgtttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttatgccacaaacacaacacgggtcggtaagaggcaggacagtgagtgggtgtcgtactcggtctacggtgagaatgttgactatgaaagataccctgtagcacatctgcaagaggaggccgacgcgtgttacgagagtttaggtaatatgattacgagccaagtacagccctgtactcagagagaatgttatgctatggatcagaaagtatgcgcagctgtcggcttctcatcagatgcgggtgttaactccgcaatggtcggtgaggcctacttctatgcctatggtggggttgatggtgaatgtgacagcggctagaataggatataaataatatattaataaaacaaaacaataaaacagaacaagtaagaacagatgaatggaatcagacggtatgtgtgggattgaaacactaaagtgtcaaaccacaacacagcaacataccgggtgtacaatgtgggtgaaggttaacaacgcctgaggtggctaagttatgcgtttcgcagactatcgctacctagactaagtaacctgagcacacatcgtaataagtgtagcatggtgatgtcgcgacacaagtggcgacaatagcgctcttagcgtacgttgcatgctggacgcaccgaaattatgaggtacatttaccctgtctcttatacacatctgacgctgccgacgatcgaacgagtgtagatctcggtgggcgccgtatcattaaaaaaaaaaaaaaaggaagaaaggttaggcgagcgcgtctaataaaagaaacaggtatacgacaagacagagaggaaggtacacggagaagtgttagagagcgcgatcactagagctataaaagaagataaa

SEQ ID NO: 33 - DNA, 1089 bp, Saccharomyces cerevisiae
atgaaagagactaccaccagcctgatgcaagacgagctgacactaggtgagccggccacccaagcaaggatgtgtgtacgtctattacgttttttcataggtctgactataaccgcatttgttatagcagcctgtattattaaaagtgcgacaggcggttcgggatattctaatgcagttgctgtttggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctattacatcgtatgtggcatatgcccctacactgtgtgctggtgcatacgttattggtgccatgagtggggcaatgtcggcaggcctcgctctgtatgctggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcaccggaattgtcaagagggattcgagacctagcatgatctcatggacggtgttgcatgacaacatgatgataacatcataccataggccagaccagctgggcgcagccgcgacagcctacaaagcttatgccacaaacacaacacgggtcggtaagaggcaggacggtgagtgggtgtcgtactcggtttacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatggtcggtgaggcctacttctatgcctatggtggggttgatggtgaatgtgacagcggctag

SEQ ID NO: 34 - DNA, 1089 bp, Saccharomyces cerevisiae
atgaaagagactaccaccagcctgatgcaagacgagctgacactaggtgagccggccacccgagcaaggatgtgcgtacgtctattacgttttttcataggtctgactataaccgcatttattatagcagcctgtattattaaaagtgcgacaggcggttcgggatattctaatgcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggcgccatgagtggggcaatgtcggcgggccttgctctgtatgccggttacaaaggatggcagtggggcggccccgggggcatggcagagagagaggacgtggcctctttttattcactactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttatgccacaaacacaacacgggtcggtaagaggcaggacggtgagtgggtgtcgtactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatggtcggtgaggcctacttctatgcttatggtggggttgatggtgaatgtgacagcggctag

SEQ ID NO: 35 - DNA, 1089 bp, Saccharomyces cerevisiae
atgaaagagactaccaccagcctgatgcaagatgagctaacactaggtgagccggccacccaagcaaggatgtgcgtacgtctattacgttttttcataggtctgactataaccgcatttattatagcagcctgtattattaaaagtgcgacaggcggttcgggatattctaatgcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcaggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacggcctacaaagcttatgccacaaacacaacacgggtcggtaagaggcaggacggtgagtgggtgtcgtactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatagtcggtgaggcctacttctatgcctatggtggggttgatggtgaatgtgacagcggctag

SEQ ID NO: 36 - DNA, 1089 bp, Saccharomyces cerevisiae
atgaaagagactaccaccagcctgatgcaagacgagctgacactaggtgagccggccacccaagcaaggatgtgcgtacgtctattacgttttttcataggtctgactataaccgcatttattatagcagcctgtattattaaaagtgcgacaggcggttcgggatattctaaagcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttataccacaaacacaacacgggtcggtaagaggcaggacggtgagtgggtgtcatactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatggtcggtgaggcctacttctatgcctatggtggggttgatggtgaatgtgacagcggctag

SEQ ID NO: 37 - DNA, 2371 bp, Saccharomyces paradoxus
gaaaaatgagaaatagtaccttcacccttaatttgatagttctgatactagtttcgagttttttcaaccacttcacgtcgggtgctgatattagtgaaaaatttggcaaaagatggagtacttcgaacacctatcatgctctagtactgggtgcgaaatacttgtgggcatgttatgaatgggaccaaccgtggcgaattgaggtagcatggtggggaacgatggctgccgaagatggggggttcattgatactatcgggtcaggtaggaacgcgtgccagatctgtctcaacaagaaagaggaccctgaaactgactgttttaccgctgtctcagaagtcttgaaaatgtctatcatgaatgctgttaagatatctataggctggcgggcacaatcagtagttaatgctgccattgataacactagtgatcaacctctgaagaggtcaaccatgccatatccatgtgatgatggctggtgccttgatagtgacaacgttacttacagcactcctgatctgtatacgcacgcagagcagttatacgcaggtacggatgtggtaatactaacaccggaatcactgacacgccgtgacagtacaggcctagaaacagtcacagtaatgaatatcacatcgggtcttactgtggacattaaccaccggatagaccacacaggttgtggaacagtatttgtgggaccaccatctacaatcgcaggggtgaacatgacactgacgaagcgcagagatgacccacatgaattcccattttgcgatggtttcataggagtggactactgcagcaatcacaattcactaggtggtatatatggtagtggggaattagatactatggcccacgaccttgcctacgctgatagtaacggtaataactatgtagacttctataagatgtactgggttaaagatagcggtaatgaagactggctaacatcattcagaacctactataccaacgcgccatccagtgctatccattggagccaatgcgatactggcgcttgagcggtctggtgtcagcgcacattaccgttcgcagtaataagtgaatatataagatcggtacgtagtatagagtaaagtgcagagccaaatatgataaaatcaaatacatacaataacataagataatgaagtattttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacaaaagaaagaaagacgtgtctaaatccgctcaccctgagcttaactggtggcactcttgagtataacacactcaccctgagtataactggtagtccacgtcacttacggactatacaaaatagtgacactacaacaataggttgggtcaaggcactgctttcagtaacgctgttttaagctaggtgattcaccagcgacatgaacaggcgcactggtcatggtgtctccaacttatttgtaatcacaagtcgatactatgtctgactagctggtgactagtaagcacttagtttcaaacttagattataaacgcagccactaaacaacgcttgtatgtttataatgtcagatttacactagacgattcgtcggtggaaaagctggcatatctgacatcaaccgtaaagtgagctccgcgttcagacagtgttataggacagccactgataagtggtgcggacaatgtatgattacgatcgatctgatgccgagacggacaggacaaataatgtcatatgtccaggagcagtattcagtagttaggcatcgttggtccaaacctgaagactatgttttgccatggtgacctgctaatacaggaacccaccaggcgggacaacctggtaagctgaccgctaacagaattaactactcagccaggccatactggtatcgctgcaatacgcgacggtagcggctatgactgttggctacaatacggatcgcggccggcgagtcaagtaacgtggctaacagcaagtacgagctgtagtccggggcatactgagatctagttcagatatgcatagtccgatcgtcactggtgacgacaataggtcctgaatactgaacaaccgcgaaacgtcatgactgagcctacagcacatctatagactcttcatgcgacgactgccgatacatcggattcgcaagtacctactctacataagtagagagtaaacgagaacaaaacaatagcaa

SEQ ID NO: 38 - protein, 340 aa, Saccharomyces paradoxus
MRNSTFTLNLIVLILVSSFFNHFTSGADISEKFGKRWSTSNTYHALVLGAKYLWACYEWDQPWRIEVAWWGTMAAEDGGFIDTIGSGRNACQICLNKKEDPETDCFTAVSEVLKMSIMNAVKISIGWRAQSVVNAAIDNTSDQPLKRSTMPYPCDDGWCLDSDNVTYSTPDLYTHAEQLYAGTDVVILTPESLTRRDSTGLETVTVMNITSGLTVDINHRIDHTGCGTVFVGPPSTIAGVNMTLTKRRDDPHEFPFCDGFIGVDYCSNHNSLGGIYGSGELDTMAHDLAYADSNGNNYVDFYKMYWVKDSGNEDWLTSFRTYYTNAPSSAIHWSQCDTGA